(19) United States
(12) Patent Application Publication
(10) Pub. No.: US 2013/0243779 A1
(43) Pub. Date: Sep. 19, 2013

(54) PEPTIDES PROTECTIVE AGAINST E. FAECALIS, METHODS AND USES RELATING THERETO

(71) Applicants: Eszter Nagy, Vienna (AT); Beatrice Tschanun, Vienna (AT); Markus Hanner, Pressbaum (AT); Manfred Berger, Wiener Neustadt (AT); Andreas Meinke, Pressbaum (AT); Alexander Von Gabain, Vienna (AT); Barbara Murray, Houston, TX (US); Kavindra Singh, Houston, TX (US)

(72) Inventors: Eszter Nagy, Vienna (AT); Beatrice Tschanun, Vienna (AT); Markus Hanner, Pressbaum (AT); Manfred Berger, Wiener Neustadt (AT); Andreas Meinke, Pressbaum (AT); Alexander Von Gabain, Vienna (AT); Barbara Murray, Houston, TX (US); Kavindra Singh, Houston, TX (US)

(73) Assignee: Intercell AG, Vienna (AT)

(21) Appl. No.: 13/147,612

(22) PCT Filed: Feb. 4, 2010
(86) PCT No.: PCT/EP2010/051341

§ 371 Date: Nov. 4, 2011

(30) Foreign Application Priority Data
Feb. 5, 2009 (EP) 09152113.8

(51) Int. Cl. C07K 14/335; C07K 16/12

(52) U.S. Cl. CPC C07K 14/335; C07K 16/1267
USPC 424/139.1; 424/190.1; 424/93.2; 435/7.1; 435/71.1; 435/331; 435/320.1; 514/21.2; 514/44 R; 530/350; 530/387.3; 530/387.9; 536/23.7

(57) ABSTRACT

The present invention relates to a protective peptide of Enterococcus faecalis (E. faecalis) or a functional active variant thereof, optionally further consisting of additional amino acid residue(s); a nucleic acid coding for the same; a pharmaceutical composition comprising said peptide or said nucleic acid; an antibody or functional active fragment thereof specifically binding to the antigen; a hybridoma cell line which produces said antibody; a method for producing said antibody; a pharmaceutical composition comprising said antibody; the use of said peptide or said nucleic acid for the manufacture of a medicament for the immunization or treatment of a subject; the use of said antibody or functional fragment thereof for the manufacture of a medicament for the treatment of an infection; a method of diagnosing an E. faecalis infection; and the use of said peptide for the isolation and/or purification and/or identification of an interaction partner of the peptide.

The present invention relates to a protective peptide of Enterococcus faecalis (E. faecalis) or a functional active variant thereof, optionally further consisting of additional amino acid residue(s); a nucleic acid coding for the same; a pharmaceutical composition comprising said peptide or said nucleic acid; an antibody or functional active fragment thereof specifically binding to the antigen; a hybridoma cell line which produces said antibody; a method for producing said antibody; a pharmaceutical composition comprising said antibody; the use of said peptide or said nucleic acid for the manufacture of a medicament for the immunization or treatment of a subject; the use of said antibody or functional fragment thereof for the manufacture of a medicament for the treatment of an infection; a method of diagnosing an E. faecalis infection; and the use of said peptide for the isolation and/or purification and/or identification of an interaction partner of the peptide.

Enterococci are gram-positive bacteria that are normal inhabitants of the alimentary tract of humans and animals. They have been recognized as a cause of infective endocarditis for more than a century {Murray, B., 1990} and have gained prominence over the last two decades as being among the most common pathogens found in hospital-acquired infections, and surgical-site infections {Richards, M. et al., 2000}. The increasing importance of enterococci as nosocomial pathogens can be attributed in part to intrinsic and acquired antibiotic resistance {Murray, B., 1990}; {Rice, L., 2001}. Treatment of multi-drug-resistant enterococcal infections poses a significant challenge to clinicians {Cetinkaya, Y. et al., 2000}; {Gold, H., 2001}, and the potential of these organisms to serve as a reservoir for antibiotic resistance genes is of great concern {French, G., 1998}; {Noble, W. et al., 1992}; {Poyart, C. et al., 1997}.

The classification of enterococci as group D streptococci dates back to the early 1930's. In 1984, enterococci were given formal genus status after several studies demonstrated a more distant relationship with the streptococci.

Enterococci are generally considered commensals of the gastrointestinal tract of a variety of organisms including man. Although there are more than 14 different species of enterococci, E. faecalis and E. faecium are the species most commonly found in humans. Several intrinsic features of Enterococcus may allow members of this genus to survive for extended periods of time, leading to its persistence and nosocomial spread. The ability of enterococci to adapt and persist in the presence of detergents may allow them to survive inadequate cleaning regimens, contributing to their persistence in the hospital. The intrinsic ruggedness of enterococci also confers an unusual level of tolerance to several classes of antibiotics including aminoglycosides, beta-lactams and quinolones. For example, the resistance of enterococci to aminoglycosides results from the ability of enterococci to block the uptake of the drug at the cell wall. Although the mechanism of high-level resistance was determined to be the result of a bifunctional enzyme {Ferretti, J. et al., 1986}, the molecular basis for the intrinsic resistance of enterococci to low-levels of aminoglycosides remains to be determined.

Among enterococci, E. faecium is unique because it is commonly used in production of fermented foods, and is also used as a probiotic bacterium. In recent years, E. faecium has been less acceptable as a food fermentation organism because of concern that this bacterium may be an intermediate host for spreading of antibiotic resistance to bacteria involved in human infections. Despite these concerns, E. faecium is still amongst the most common bacteria found in foods fermented by lactic acid bacteria. Many isolates of E. faecium have been shown to produce bacteriocins (antimicrobial peptides) that are able to kill or inhibit growth of pathogens such as Listeriae, Clostridia, bacilli, and staphylococci. Such bacteriocins may contribute to the preservative effect of food fermentations, and is one reason why they have been chosen as starter cultures in the production of fermented food. Recently, enterocins have been implemented successfully in treatment of mastitis in cattle.

Besides the applications for food production, as probiotics and in treatment of animal disease, and more importantly, enterococci are emerging opportunistic human pathogens. This is due to their intrinsic pathogenic potential, and, even more because of their ability to rapidly acquire antibiotic resistance genes. E. faecium and E. faecalis are the causing agents of a large percentage of hospital-acquired infections, including super infections.

Enterococci normally colonize the gastrointestinal tract of man. They are found in relative abundance in human feces. A close association is likely to exist between enterococci and its host, or the organism would be eliminated due to normal intestinal motility. Many infection-derived enterococcal isolates were found to be clonal, indicating nosocomial transmission. Moreover, a number of studies have documented patient colonization following hospital admission, and have shown that colonization with multiple resistant strains is a predisposing factor for subsequent infection.

One of the enigmas of nosocomial enterococcal infection not easily explained is the ready colonization of an ecology already occupied by members of the same species. As noted, antibiotics lacking substantial anti-enterococcal activity (i.e. antibiotics that do not deleteriously affect indigenous enterococci) are important predisposing factors for infection. These infections are frequently caused by multiple resistant enterococcal isolates that have been exogenously acquired and appear to have out competed indigenous enterococci in the absence of direct selection. The fact that exogenous, multiple resistant, nosocomially transmitted enterococci efficiently colonize the gastrointestinal tract suggests that they may not compete directly for the same niche as indigenous strains.

Infection caused by the genus Enterococcus include a) bacteremia, b) urinary tract infections c) endophthalmitis, d) endocarditis and also wound and intra-abdominal infections. Approximately 75% of the infections are caused by the species E. faecalis, the rest by E. faecium:

### a) Bacteremia

Nosocomial surveillance data for the period October 1986-April 1997 list enterococci as the third most common cause of nosocomial bacteremia, accounting for 12.8% of all isolates. The translocation of enterococci across an intact intestinal epithelial barrier is thought to lead to many bacteremias with no identifiable source. The risk factors for mortality associated with enterococcal bacteremia include severity of illness, patient age, and use of broad spectrum antibiotics, such as third-generation cephalosporins or metronidazole. These studies suggest that high-level aminoglycoside resistance does not affect clinical outcome, and that the presence of the E. faecalis cytolysin (hemolysin) may enhance the severity of the infection.

### b) Urinary Tract Infections

Enterococci have been estimated to account for 110,000 urinary tract infections (UTI) annually in the United States. A few studies have been aimed at understanding the interaction of enterococci with uroepithelial tissue. A potential role for the plasmid-encoded aggregation substance in the adhesion of enterococci to renal epithelial cells has been demonstrated. E. faecalis harboring the pheromone responsive plasmid pAD1, or various isogenic derivatives, were better able to bind to the cultured pig renal tubular cell line, LLC-PK, than plasmid free cells. Their findings also showed that a synthetic peptide containing the fibronectin motif, Arg-Gly-Asp-Ser, could inhibit binding. This structural motif mediates the interaction between fibronectin and eukaryotic surface receptors of the integrin family.

### c) Endophthalmitis

Colonization of host tissue may play a role in the pathogenesis of endophthalmitis. Enterococci are among the most destructive agents that cause this post-operative complication of cataract surgery. Experiments designed to determine whether aggregation substance targeted E. faecalis to alternate anatomical structures within the eye showed that enterococci attach to membranous structures in the vitreous, but that such adherence is not dependent on the presence of aggregation substance.

### d) Endocarditis

Of the diverse infections caused by enterococci, infective endocarditis (IE) is one of the most therapeutically challenging. Enterococci are the third leading cause of infective endocarditis, accounting for 5-20% of cases of native valve IE, and 6-7% of prosthetic valve endocarditis. The presence of the pheromone-responsive plasmid pAD1 enhances vegetation formation in enterococcal endocarditis. Serum from a patient with E. faecalis endocarditis was used to identify an antigen selectively expressed in serum but not in broth culture {Lowe, A. et al., 1995}. This protein antigen, designated EfaA, has extensive sequence similarity with several streptococcal adhesions and might function as an important adhesin in endocarditis.

Ampicillin is the therapy of choice for enterococcal infections. For serious enterococcal infection, particularly for endocarditis, aminoglycosides are critical as part of combination therapy with penicillin or ampicillin. Although enterococci are intrinsically resistant to low levels of aminoglycosides, the addition of the cell wall inhibitors to aminoglycoside will result in an enhanced killing by the synergistic action of the two antimicrobials. With the increasing incidence of high level resistance to aminoglycosides and penicillins, vancomycin has become the only choice available for the treatment of enterococcal infections. Then, vancomycin resistance was reported in clinical isolates of enterococci in 1988, followed by an outbreak caused by vancomycin-resistant enterococci (VRE). In U.S. hospitals the percentage of nosocomial enterococci resistant to vancomycin increased from 0.3% in 1989 to 7.9% in 1993. Among patients in intensive care units with nosocomial infections an increase was even more dramatic; from 0.4% in 1989 to 13.6% in 1993, a 34-fold increase in the 4-year period.

In addition to a higher mortality rate, vancomycin-resistant enterococcal infections cost on average about $25,000 more to treat and doubled the patients' length of stay in the hospital.

The dramatic increase in vancomycin resistance, especially among E. faecium isolates, indicates that enterococcal infection will pose an increasing challenge in the future. An obvious therapeutic alternative is vaccination with the aim to induce protective immune responses, which prevents or attenuates infections.

Vaccine development is hindered by the lack of sufficient knowledge about the elements of protective immunity against enterococcal infections. There are reports that neutrophil mediated killing of enterococci was largely a function of complement with antibody playing a less essential but potentially important role, though additional evidence for the importance of anti-enterococcal antibodies in promoting clearance by opsonophagocytic killing was recently reported {Gaglani, M. et al., 1997}.

The importance of surface proteins in human immunity to Enterococcus already has been appreciated. It is apparent that all clinical isolates express surface proteins with activity relevant to host immune defense. The enterococcal surface protein (Esp) {Shankar, V. et al., 1999}, gelatinase, cytolysin {Haas, W. et al., 2002} and aggregation substance (AS) surface protein {Sussmuth, S. et al., 2000} are well-characterized biochemically and genetically, and have also been shown to be immunogenic {Xu, Y. et al., 1997}. In an animal model of infective endocarditis specific antibodies against the aggregation substance were still not protective {McCormick, J. et al., 2001}.

Thus, there remains a need for an effective treatment to prevent or ameliorate enterococcal infections. Vaccines capable of showing cross-protection against the majority of Enterococcus strains causing human infections could also be useful to prevent or ameliorate infections caused by all other enterococcal species, namely E. faecalis and E. faecium.

A vaccine can contain a whole variety of different antigens. Examples of antigens are whole-killed or attenuated organisms, subfractions of these organisms/tissues, proteins, or, in their most simple form, peptides. Antigens can also be recognized by the immune system in form of glycosylated proteins or peptides and may also be or contain polysaccharides or lipids. Short peptides can be used since for example cytotoxic T-cells (CTL) recognize antigens in form of short usually 8-11 amino acids long peptides in conjunction with major histocompatibility complex (MHC). B-cells can recognize linear epitopes as short as 4-5 amino acids, as well as three-dimensional structures (conformational epitopes). In order to obtain sustained, antigen-specific immune responses, adjuvants need to trigger immune cascades that involve all cells of the immune system necessary. Primarily, adjuvants are acting, but are not restricted in their mode of action, on so-called antigen presenting cells (APCs). These cells usually first encounter the antigen(s) followed by presentation of processed or unmodified antigen to immune effector cells. Intermediate cell types may also be involved. Only effector cells with the appropriate specificity are activated in a productive immune response. The adjuvant may also locally retain antigens and co-injected other factors. In addition the adjuvant may act as a chemoattractant for other immune cells or may act locally and/or systemically as a stimulating agent for the immune system.

Currently vaccines against enterococcal infection are only in the research stages of development. Efforts are focused not only on capsular polysaccharide (CPS) as immunogens {Huebner, J. et al., 2000}, but also on virulence factors and membrane/surface proteins.

The development of protein conjugated vaccines are no doubt a great new addition to the amarmatorium in the battle against enterococcal infections, but the vaccine can contain only a limited number of enterococcal proteins and given adequate ecological pressure, variation of the pathogenicity island and plasmids by non-vaccine clinical isolates remains a real threat. Moreover polysaccharide antigens used for active immunization do not provide immunological memory in humans. Conjugation of CPS to non-enterococcal related immunogenic protein carriers (e.g. tetanus toxoid, cholera toxin B subunit, etc.) has been shown to be beneficial in inducing higher concentrations of antibodies in vaccinees, but it does not provide pathogen-specific B cell and T cell epitopes which would recruit memory B and T cells during a real infection to support the most effective host response. To be able to supplement the enterococcal vaccines with proteins fulfilling these criteria it is necessary to identify conserved immunogenic enterococcal-specific surface proteins.

Certain proteins or enzymes displayed on the surface of gram-positive organisms significantly contribute to pathogenesis, are involved in the disease process caused by these pathogens. Often, these proteins are involved in direct interactions with host tissues or in concealing the bacterial surface from the host defense mechanisms {Navarre, W. et al., 1999}. E. faecalis is not an exception in this regard. Several surface proteins are characterized as virulence factors, important for enterococcal pathogenicity reviewed in {Jett, B. et al., 1994}. If antibodies to these proteins could offer better protection to humans then polysaccharides, they could provide the source of a novel, protein-based enterococcal vaccine to be used in conjunction with or in place of the more traditional capsular polysaccharide vaccine. The use of some of the above-described proteins as antigens for a potential vaccine is based on easiness of identification or chance of availability.

In order to identify vaccine candidates from bacterial pathogens methods for identification, isolation and production of hyperimmune serum reactive antigens from a specific pathogen, especially from Staphylococcus aureus and Staphylococcus epidermidis, have been developed (WO 02/059148). Moreover, methods for identification of reactive antigens as well as reactive antigens from E. faecalis have been provided (WO 04/106367).

However, some of these antigens are large proteins, which may be disadvantageous in the context of immunization. First, providing of smaller antigens eases production of the protein in recombinant form. It is generally accepted that it is more difficult to produce larger proteins. Smaller proteins may be produced in a more economic manner, thus saving costs, particularly in the health care system. Second, reducing the size of antigenic proteins used for vaccination may lead to safer products. Eliminating extra sequences in antigenic proteins is desirable, since this reduces the probability of inducing antibodies which can cause cross-reactions with human tissues. Third, proteins used for vaccination may contain more than one antigen, the antigens directed either against the same disease or against different diseases, in order to obtain a more effective vaccination or vaccination against several diseases. However, if the single antigens are too large a combination into one fusion protein is not feasible.

On the other hand, there is a great potential for passive antibody-based therapy. There have been already attempts to use human intravenous immunoglobulin (IVIG) preparations for prevention. Recent advances in the technology of monoclonal antibody production provide the means to generate human antibody reagents and reintroduce antibody therapies, while avoiding the toxicities associated with serum therapy. Immunoglobulins are an extremely versatile class of antimicrobial proteins that can be used to prevent and treat emerging infectious diseases. Antibody therapy has been effective against a variety of diverse microorganisms reviewed in {Burnie, J. et al., 1998}. Anti-enterococcal mAB could be given therapeutically to immunosuppressed patient, due to organ transplantation, cancer, HIV infection and other causes.

Accordingly, one problem underlying the present invention was to provide an appropriate antigen of E. faecalis for the development of medicaments such as vaccines against an Enterococcus infection. More particularly, the problem was to provide a protective peptide or combinations thereof from E. faecalis that can be used for the manufacture of said medicaments.

Consequently, the present invention provides a protective peptide of a hyperimmune serum reactive antigen consisting of the amino acid sequence of the SEQ ID NO: 21, 22, 23, 24, 25, 26, 27, 28, 29, 30, 31, 32, 33, 34, 35, 36, 37, 38, 39 or 40, or a functional active variant of the protective peptide, or a fragment (portion) thereof. These peptides are also referred to as antigenic peptides.

The protective peptides consisting of the amino acid sequences of SEQ ID NOs: 21 to 40 are derived from E. faecalis strain V583 described earlier (GenBank accession numbers for chromosome and the plasmids are: AE016830 (chromosome), AE016833 (pTEF1), AE016831 (pTEF2), AE016832 (pTEF3); {Paulsen, I. et al., 2003}).

The amino acid and DNA sequences of the full length antigens from which the protective peptides consisting of the amino acid sequences of SEQ ID NOs: 21 to 40 are derived are disclosed in WO 04/106367.

The amino acid sequences of SEQ ID NOs: 21 to 40 and corresponding DNA sequences of SEQ ID NOs: 1 to 20 are disclosed in the attached Sequence Listing and Table 2 (see Example 1). The peptides of SEQ ID NOs: 21 to 40 have been shown to induce protective immune response against E. faecalis in an animal model of infective endocarditis (see Example 1 and Figures). Functional active variants may be obtained by changing the sequence of the protective peptide as defined below and are characterized by having a biological activity similar to that displayed by the protective peptide of the sequence of SEQ ID NOs: 21 to 40 from which the variant is derived, including the ability to induce protective immune responses and/or to show protection against E. faecalis e.g. in an animal model of infective endocarditis, wherein any variant may be tested as described in the Examples.

In another preferred embodiment of the invention the peptide of the invention or a functional active variant, can further consist of
a) 1 to 450 additional amino acid residue(s), preferably 1 to 400, 1 to 350, 1 to 300, 1 to 250, 1 to 200, 1 to 150, more preferably 1 to 100, even more preferably at most 1 to 50, most preferably 1, 2, 3, 4, 5, 10, 20, 30 or 40 additional amino acids residue(s) if the antigen is SEQ ID NO: 21; or
b) 1 to 400 additional amino acid residue(s), preferably 1 to 350, 1 to 300, 1 to 250, 1 to 200, 1 to 150, more preferably 1 to 100, even more preferably at most 1 to 50, most preferably 1, 2, 3, 4, 5, 10, 20, 30 or 40 additional amino acids residue(s) if the antigen is SEQ ID NO: 22; or
c) 1 to 300 additional amino acid residue(s), preferably 1 to 250, 1 to 200, 1 to 150, more preferably 1 to 100, even more preferably at most 1 to 50, most preferably 1, 2, 3, 4, 5, 10, 20, 30 or 40 additional amino acids residue(s) if the antigen is SEQ ID NO: 24; or
d) 1 to 125 additional amino acid residue(s), preferably 1 to 100, 1 to 80, 1 to 60, more preferably 1 to 40, even more preferably at most 1 to 30, most preferably 1, 2, 3, 4, 5, 10, 15, 20, or 25 additional amino acids residue(s) if the antigen is SEQ ID NO: 25.

The protective peptide of E. faecalis can be any of the peptides as defined above, namely as defined in any of the SEQ ID NO: 21, 22, 23, 24, 25, 26, 27, 28, 29, 30, 31, 32, 33, 34, 35, 36, 37, 38, 39 or 40, or a functional active variant thereof, wherein the functional active variant is as defined below.

The functional active variant of the protective peptide may have added at least one additional amino acid residue heterologous or homologous to the peptide of any of the SEQ ID NOs: 21 to 40. Homologous refers to any amino acid residue which is identical to the amino acid residue of the protein from E. faecalis from which the peptide is derived, wherein the peptide of any of the SEQ ID NO: 21 to 40 is derived from the protein as listed in the table below (Table 1). Alternatively or additionally, the protective peptide may consist of the antigen, optionally the additional sequence as defined above and at least one amino acid residue heterologous to the antigen, preferably a marker protein.

TABLE 1
Peptides of the present invention are related to proteins from
WO 04/106367.

| | Derived from protein (as disclosed in WO |
|---|---|
| Protective peptide | 04/106367) |
| SEQ ID NO: 21 | EF0032 |
| SEQ ID NO: 22 | EF0270 |
| SEQ ID NO: 23 | EF0428 |
| SEQ ID NO: 24 | EF0779 |
| SEQ ID NO: 25 | EF0792 |
| SEQ ID NO: 26 | EF1026 |
| SEQ ID NO: 27 | EF1032 |
| SEQ ID NO: 28 | EF1060 |
| SEQ ID NO: 29 | EF1093 |
| SEQ ID NO: 30 | EF1277 |
| SEQ ID NO: 31 | EF1355 |
| SEQ ID NO: 32 | EF1692 |
| SEQ ID NO: 33, 41 | EF2703 |
| SEQ ID NO: 34 | EF3051 |
| SEQ ID NO: 35 | EF3177 |
| SEQ ID NO: 36 | EF3183 |
| SEQ ID NO: 37, 42 | EF3207 |
| SEQ ID NO: 38, 43 | EF3256 |
| SEQ ID NO: 39 | EFA0042 |
| SEQ ID NO: 40, 44 | EFC0034 |

The functional active variant of the protective peptide may be obtained by sequence alterations in the protective peptide, wherein the peptide with the sequence alterations retains a function of the unaltered protective peptide, e.g. having a biological activity similar to that displayed by the unaltered protective peptide. Such sequence alterations can include, but are not limited to, (conservative) amino acid substitutions, deletions, mutations and insertions.

The additional sequence or amino acid residue(s) as defined above consists of (an) amino acid residue(s), which may be any amino acid, which may be either an L- and/or a D-amino acid, naturally occurring and otherwise. Preferably the amino acid is any naturally occurring amino acid such as alanine, cysteine, aspartic acid, glutamic acid, phenylalanine, glycine, histidine, isoleucine, lysine, leucine, methionine, asparagine, proline, glutamine, arginine, serine, threonine, valine, tryptophan or tyrosine.

However, the amino acid may also be a modified or unusual amino acid. Examples of those are 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproloine, 4-hydroxyproloine, isodesmosine, allo-isoleucine, N-methylglycine, N-methylisoleucine, 6-N-Methyllysine, N-methylvaline, norvaline, norleucine or ornithine. Additionally, the amino acid may be subject to modifications such as posttranslational modifications. Examples of modifications include acetylation, amidation, blocking, formylation, γ-carboxyglutamic acid hydroxylation, glycosilation, methylation, phosphorylation and sulfatation. If more than one additional or heterologous amino acid residue is present in the peptide, the amino acid residues may be the same or different from one another.

In one embodiment the peptide of the invention further encompasses at least one amino acid residue heterologous to the antigen. The feature “heterologous amino acid” or “amino acid heterologous to the antigen or protein” refers to any amino acid which is different from that amino acid located adjacent to the antigen or protein in any naturally occurring protein of E. faecalis, particularly from that of E. faecalis strain V583 (serogroup CPS-C). Therefore, the protein of the invention encompassing at least one heterologous amino acid refers to a protein which is different from any naturally occurring protein of E. faecalis or fragment thereof, particularly which is different from that of E. faecalis strain V583 (serogroup CPS-C). The proteins from which the antigens of the invention are derived as well as a reference for their sequences are disclosed in WO 04/106367 and in the table above (Table 1).

In one preferred embodiment of the invention the peptide of the invention comprising additional amino acid residue(s) as defined above is characterized in that it comprises at least 2, preferably at least 3, more preferably at least 4 antigens as defined above.

The antigenic peptide may be flanked by the amino acid residue(s) C-terminally, N-terminally or C- and N-terminally.

In one embodiment of the invention, the functional active variant of the peptide of the invention is essentially identical to any of the antigens of the SEQ ID NOs: 21 to 40, but differs from the antigens of any of the of the SEQ ID NOs: 21 to 40, in that it is derived from a homologous sequence of a different strain and/or serotype of E. faecalis.

Accordingly, the present invention also relates to antigens of different E. faecalis isolates. Such homologues may easily be isolated based on the nucleic acid and amino acid sequences disclosed herein. The presence of any antigen can be determined for every serotype. In addition it is possible to determine the variability of a particular antigen in the various serotypes as described for the sic gene {Hoe, N. et al., 2001}. The contribution of the various serotypes to the different enterococcal infections varies in the different age groups and geographical regions. It is an important aspect that the most valuable protective antigens are expected to be conserved among various clinical isolates. A homologous antigen of a different serotype may be identified by e.g. sequence alignment. The homologous antigen sequence may vary from the antigen of any of the sequences of SEQ ID NOs: 21 to 40 by one or more amino acid substitutions, deletions and/or additions.

In another embodiment of the present invention the functional active variant is a fragment of any of the sequences of SEQ ID NOs: 21 to 40. The fragment is characterized by being derived from the antigen as defined above by one or more amino acid deletions. The deletion(s) may be C-terminally, N-terminally and/or internally. Preferably the fragment is obtained by at most 10, 20, 30, 40, 50, 60, 80, 100, 150 or 200, more preferably by at most 10, 20, 30, 40 or 50, even more preferably at most 5, 10 or 15, still more preferably at most 5 or 10, most preferably 1, 2, 3, 4 or 5 deletion(s). The functional active fragment of the invention is characterized by having a biological activity similar to that displayed by the complete antigen, including the ability to induce immune response and/or to show protection against E. faecalis e.g. in an infective endocarditis model. The fragment of an antigen is functional active in the context of the present invention, if the activity of the fragment amounts to at least 10%, preferably at least 25%, more preferably at least 50%, even more preferably at least 70%, still more preferably at least 80%, especially at least 90%, particularly at least 95%, most preferably at least 99% of the activity of the antigen without sequence alteration. These fragments may be designed or obtained in any desired length, including as small as about 50 to 80 amino acids in length.

In a further embodiment a fragment of the peptide of the invention are characterized by structural or functional attributes, such as alpha-helix, beta-sheet, turn and coil-forming regions, hydrophilic regions, hydrophobic regions, alpha-amphipathic regions, beta-amphipathic regions, flexible regions, surface-forming regions, substrate binding regions, and high antigenic index regions of the peptide according to the present invention, and combinations of such fragments. Preferred regions are those that mediate antigenicity and antibody binding activities of the peptides according to the present invention. Most highly preferred in this regard are fragments that have a chemical, biological or other activity of the peptides and variants thereof of the present invention, including those with a similar activity or an improved activity, whereby such improved activities are immunogenicity and stability, or with a decreased undesirable activity, whereby such decreased undesirable activity is enzymatic and toxic function and generation of human cross-reactive antibodies. Particularly preferred fragments of the peptides according to the present invention are those that comprise or contain antigenic or immunogenic determinants in an animal, especially in a human. Such fragments are also referred to as antigenic fragment. An antigenic fragment is antigenic by itself or may be made antigenic when provided as a hapten.

Accordingly, in one preferred embodiment of the invention the functional active fragments consists of at least 60%, preferably at least 70%, more preferably at least 80%, still more preferably at least 90%, even more preferably at least 95%, most preferably 99% of the antigenic peptide of any of the SEQ ID NOs: 21 to 40.

Another preferred embodiment of the invention relates to a peptide as defined above in the previous embodiments, wherein the peptide is a functional active variant of an antigen of any of the SEQ ID NOs: 21 to 40 and wherein the variant has at least 50% sequence identity to the antigen of any of the SEQ ID NOs: 21 to 40. In a more preferred embodiment the functional active variant has a sequence identity of at least 60%, preferably at least 70%, more preferably at least 80%, still more preferably at least 90%, even more preferably at least 95%, most preferably 99% to the antigen of any of the SEQ ID NOs: 21 to 40.

The percentage of sequence identity can be determined e.g. by sequence alignment. Methods of alignment of sequences for comparison are well known in the art. Various programs and alignment algorithms have been described e.g. in Smith and Waterman, Adv. Appl. Math. 2: 482, 1981 or Pearson and Lipman, Proc. Natl. Acad. Sci. U.S.A. 85: 2444-2448, 1988.

The NCBI Basic Local Alignment Search Tool (BLAST) (Altschul et al., J. Mol. Biol. 215: 403-410, 1990) is available from several sources, including the National Center for Biotechnology Information (NCBI, Bethesda, Md.) and on the Internet, for use in connection with the sequence analysis programs blastp, blastn, blastx, tblastn and tblastx. Variants of an antigen of any of the sequences of SEQ ID NOs: 21 to 40 are typically characterized using the NCBI Blast 2.0, gapped blastp set to default parameters. For comparisons of amino acid sequences of at least 35 amino acids, the Blast 2 sequences function is employed using the default BLOSUM62 matrix set to default parameters, (gap existence cost of 11, and a per residue gap cost of 1). When aligning short peptides (fewer than around 35 amino acids), the alignment is performed using the Blast 2 sequences function, employing the PAM30 matrix set to default parameters (open gap 9, extension gap 1 penalties). Methods for determining sequence identity over such short windows such as 15 amino acids or less are described at the website that is maintained by the National Center for Biotechnology Information in Bethesda, Md. (http://www.ncbi.nlm.nih.gov/BLAST/).

The functional active variant of an antigen is obtained by sequence alterations in the antigen, wherein the antigen with the sequence alterations retains a function of the unaltered antigen. Such sequence alterations can include, but are not limited to, conservative substitutions, deletions, mutations and insertions. In the context of the present invention a variant specifically has a biological activity similar to that displayed by the antigen without alteration, including the ability to induce an immune response and/or to show protection against E. faecalis e.g. in an infective endocarditis model if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of the antigen without sequence alterations.

The term “functional active variant” includes naturally-occurring allelic variants, as well as mutants or any other non-naturally occurring variants. As is known in the art, an allelic variant is an alternate form of a (poly)peptide that is characterized as having a substitution, deletion, or addition of one or more amino acids that does essentially not alter the biological function of the polypeptide. By “biological function” is meant a function of the polypeptide in the cells in which it naturally occurs, even if the function is not necessary for the growth or survival of the cells. For example, the biological function of a porin is to allow the entry into cells of compounds present in the extracellular medium. The biological function is distinct from the antigenic function. A polypeptide can have more than one biological function.

Within any species of the living world, allelic variation is the rule. For example, any bacterial species, e.g. E. faecalis, is usually represented by a variety of strains (characterized by clonal reproduction) that differ from each other by minor allelic variations. Indeed, a polypeptide that fulfils the same biological function in different strains can have an amino acid sequence that is not identical in each of the strains. Such an allelic variation is equally reflected at the polynucleotide level.

In a preferred embodiment, the functional active variant or fragment derived from the antigen by amino acid exchanges, deletions or insertions may also conserve, or more preferably improve, the activity (as defined above). Furthermore, these peptides may also cover epitopes, which trigger the same or preferably an improved T cell response. These epitope are referred to as “heteroclitic”. They have a similar or preferably greater affinity to MHC/HLA molecules, and the ability to stimulate the T cell receptors (TCR) directed to the original epitope in a similar or preferably stronger manner. Heteroclitic epitopes can be obtained by rational design i.e. taking into account the contribution of individual residues to binding to MHC/HLA as for instance described by (Rammensee, H. et al., 1999, Immunogenetics. 50: 213-219), combined with a systematic exchange of residues potentially interacting with the TCR and testing the resulting sequences with T cells directed against the original epitope. Such a design is possible for a skilled man in the art without much experimentation.

In a still more preferred embodiment of the invention the functional active variant of an antigen of any of the SEQ ID NOs: 21 to 40 having at least 50% sequence identity to the antigen of any of the SEQ ID NOs: 21 to 40, especially at least 60%, preferably at least 70%, more preferably at least 80%, still more preferably at least 90%, even more preferably at least 95%, most preferably 99% to the antigen of any of the SEQ ID NOs: 21 to 40 is derived from the antigen of any of the sequences of SEQ ID NOs: 21 to 40 by conservative substitutions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. In one embodiment, one conservative substitution is included in the peptide. In another embodiment, two conservative substitutions or less are included in the peptide. In a further embodiment, three conservative substitutions or less are included in the peptide.

Examples of conservative amino acid substitutions include, but are not limited to, those listed below:

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| His | Asn; Gln |
| Ile | Leu; Val |
| Leu | Ile; Val |
| Lys | Arg; Gln; Asn |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

In another embodiment of the invention the peptide as defined above may be modified by a variety of chemical techniques to produce derivatives having essentially the same activity (as defined above for fragments and variants) as the modified peptides, and optionally having other desirable properties. For example, carboxylic acid groups of the protein, whether C-terminal or side chain, may be provided in the form of a salt of a pharmaceutically-acceptable cation or esterified to form an ester, or converted to an amide. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be converted to an amide. Hydroxyl groups of the peptide side chains may be converted to alkoxy or to an ester using well recognized techniques. Phenyl and phenolic rings of the peptide side chains may be substituted with one or more halogen atoms, such as fluorine, chlorine, bromine or iodine, or with alkyl, alkoxy, carboxylic acids and esters thereof, or amides of such carboxylic acids. Thiols can be protected with any one of a number of well recognized protecting groups, such as acetamide groups.

Peptides of this invention may be in combination with outer surface proteins or other proteins or antigens of other proteins. In such combination, the antigen may be in the form of a fusion protein. The antigen of the invention may be optionally fused to a selected peptide or protein derived from other microorganisms. For example, an antigen or polypeptide of this invention may be fused at its N-terminus or C-terminus to a polypeptide from another pathogen or to more than one polypeptide in sequence. Peptides which may be useful for this purpose include polypeptides identified by the prior art.

In a preferred embodiment of the invention the peptide of the invention is fused to an epitope tag which provides an epitope to which an anti-tag substance can selectively bind. The epitope tag is generally placed at the amino- or carboxyl-terminus of the peptide but may be incorporated as an internal insertion or substitution as the biological activity permits. The presence of such epitope-tagged forms of a peptide can be detected using a substance such as an antibody against the tagged peptide. Also, provision of the epitope tag enables the peptide to be readily purified by affinity purification using an anti-tag antibody or another type of affinity matrix that binds to the epitope tag. Various tag polypeptides and their respective antibodies are well known in the art. Examples include poly-histidine (poly-his), poly-histidine-glycine (poly-his-gly) tags, the HA tag polypeptide, the c-myc tag, the Strep tag and the FLAG tag.

Fusions also may include the peptides or antigens of this invention fused or coupled to moieties other than amino acids, including lipids and carbohydrates. Further, antigens of this invention may be employed in combination with other vaccinal agents described by the prior art, as well as with other species of vaccinal agents derived from other microorganisms. Such proteins are useful in the prevention, treatment and diagnosis of diseases caused by a wide spectrum of Streptococcus isolates.

These fusion proteins are constructed for use in the methods and compositions of this invention. These fusion proteins or multimeric proteins may be produced recombinantly, or may be synthesized chemically.

The peptides of the invention may be prepared by any of a number of conventional techniques. Desired peptides may be chemically synthesized. An alternative approach involves generating the fragments of known peptides by enzymatic digestion, e.g., by treating the protein with an enzyme known to cleave proteins at sites defined by particular amino acid residues, or by digesting the DNA with suitable restriction enzymes, expressing the digested DNA and isolating the desired fragment. Yet another suitable technique involves isolating and amplifying a DNA fragment encoding a desired peptide fragment, by polymerase chain reaction (PCR). Oligonucleotides that define the desired termini of the DNA fragment are employed as the 5′ and 3′ primers in the PCR. Techniques for making mutations, such as deletions, insertions and substitutions, at predetermined sites in DNA, and therefore in proteins, having a known sequence are well known. One of skill in the art using conventional techniques, such as PCR, may readily use the antigens and peptides provided herein to identify and isolate other similar proteins. Such methods are routine and not considered to require undue experimentation, given the information provided herein. For example, variations can be made using oligonucleotide-mediated site-directed mutagenesis (Carter et al., Nucl. Acids Res., 13: 4431 (1985); Zoller et al., Nucl. Acids Res. 10:6487 (1987)), cassette mutagenesis (Wells et al., Gene, 34:315 (1985)), restriction selection mutagenesis (Wells et al., Philos. Trans. R. Soc. London SerA, 317:415 (1986)), PCR mutagenesis, or other known techniques can be performed on the cloned DNA to produce the peptide of the invention.

Another subject of the present invention relates to a nucleic acid encoding a peptide of the invention, i.e. any peptide as defined above, or a nucleic acid complementary thereto. Nucleic acid molecules of the present invention may be in the form of RNA, such as mRNA or cRNA, or in the form of DNA, including, for instance, cDNA and genomic DNA e.g. obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand. Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or a mixture of single- and double-stranded regions.

The nucleic acid may be a fragment of a nucleic acid occurring naturally in E. faecalis. Preferably the nucleic acid has a sequence as defined in any of the sequences of SEQ ID NOs: 1 to 20 or of any of the homologous variants identified in the attached listing of nucleic acid sequences. The nucleic acid also includes sequences that are a result of the degeneration of the genetic code. There are 20 natural amino acids, most of which are specified by more than one codon. Therefore, all nucleotide sequences are included in the invention which result in the peptide as defined above.

Additionally, the nucleic acid may contain one or more modified bases. Such nucleic acids may also contain modifications e.g. in the ribose-phosphate backbone to increase stability and half life of such molecules in physiological environments. Thus, DNAs or RNAs with backbones modified for stability or for other reasons are “nucleic acid molecule” as that feature is intended herein. Moreover, DNAs or RNAs comprising unusual bases, such as inosine, or modified bases, such as tritylated bases, to name just two examples, are nucleic acid molecule within the context of the present invention. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia. For example, nucleotide substitutions can be made which do not affect the polypeptide encoded by the nucleic acid, and thus any nucleic acid molecule which encodes an antigen or fragment or functional active variant thereof as defined above is encompassed by the present invention.

Furthermore, any of the nucleic acid molecules encoding an antigen of the invention or fragment or functional active variant thereof can be functionally linked, using standard techniques such as standard cloning techniques, to any desired regulatory sequences, whether an E. faecalis regulatory sequence or a heterologous regulatory sequence, heterologous leader sequence, heterologous marker sequence or a heterologous coding sequence to create a fusion protein.

The nucleic acid of the invention may be originally formed in vitro or in a cell in culture, in general, by the manipulation of nucleic acids by endonucleases and/or exonucleases and/or polymerases and/or ligases and/or recombinases or other methods known to the skilled practitioner to produce the nucleic acids.

In one embodiment of the invention the nucleic acid is located in a vector. A vector may additionally include nucleic acid sequences that permit it to replicate in the host cell, such as an origin of replication, one or more therapeutic genes and/or selectable marker genes and other genetic elements known in the art such as regulatory elements directing transcription, translation and/or secretion of the encoded protein. The vector may be used to transduce, transform or infect a cell, thereby causing the cell to express nucleic acids and/or proteins other than those native to the cell. The vector optionally includes materials to aid in achieving entry of the nucleic acid into the cell, such as a viral particle, liposome, protein coating or the like. Numerous types of appropriate expression vectors are known in the art for protein expression, by standard molecular biology techniques. Such vectors are selected from among conventional vector types including insects, e.g., baculovirus expression, or yeast, fungal, bacterial or viral expression systems. Other appropriate expression vectors, of which numerous types are known in the art, can also be used for this purpose. Methods for obtaining such expression vectors are well-known (see, e.g. Sambrook et al., Molecular Cloning. A Laboratory Manual, 2^{nd} edition, Cold Spring Harbor Laboratory, New York (1989)). In one embodiment, the vector is a viral vector. Viral vectors include, but are not limited to, retroviral and adenoviral vectors.

Suitable host cells or cell lines for transfection by this method include bacterial cells. For example, the various strains of E. coli are well-known as host cells in the field of biotechnology. Various strains of B. subtilis, Pseudomonas, Streptomyces, and other bacilli and the like may also be employed in this method. Many strains of yeast cells known to those skilled in the art are also available as host cells for expression of the peptides of the present invention. Other fungal cells such as Aspergillus cells or insect cells such as Spodoptera frugipedera (Sf9) or Drosophila S2 cells may also be employed as expression systems. Alternatively, mammalian cells, such as human 293 cells, Chinese hamster ovary cells (CHO), the monkey COS-1 or murine 3T3 cell lines, HeLa, C127, BHK, 293, Bowes melanoma cells or plant cells may be used. Still other suitable host cells, as well as methods for transfection, culture, amplification, screening, production, and purification are known in the art.

A peptide of the invention may be produced by expressing a nucleic acid of the invention in a suitable host cell. The host cells can be transfected, e.g. by conventional means such as electroporation with at least one expression vector containing a nucleic acid of the invention under the control of a transcriptional regulatory sequence. The transfected or transformed host cell is then cultured under conditions that allow expression of the protein. The expressed protein is recovered, isolated, and optionally purified from the cell (or from the culture medium, if expressed extracellularly) by appropriate means known to one of skill in the art. For example, the proteins are isolated in soluble form following cell lysis, or extracted using known techniques, e.g. in guanidine chloride. If desired, the peptides or fragments of the invention are produced as a fusion protein. Such fusion proteins are those described above. Alternatively, for example, it may be desirable to produce fusion proteins to enhance expression of the protein in a selected host cell or to improve purification. The molecules comprising the peptides and antigens of this invention may be further purified using any of a variety of conventional methods including, but not limited to: liquid chromatography such as normal or reversed phase, using HPLC, FPLC and the like; affinity chromatography (such as with inorganic ligands or monoclonal antibodies); size exclusion chromatography; immobilized metal chelate chromatography; gel electrophoresis; and the like. One of skill in the art may select the most appropriate isolation and purification techniques without departing from the scope of this invention. Such purification provides the antigen in a form substantially free from other proteinaceous and non-proteinaceous materials of the microorganism.

As an alternative, a peptide of the preset invention may be produced in cell-free translation systems using RNAs derived from the DNA construct of the present invention. Otherwise, the peptides of the invention can be synthetically produced by conventional peptide synthesizers.

Another subject of the invention is a pharmaceutical composition, preferably a vaccine, comprising at least one protective peptide according to the invention and optionally a pharmaceutically acceptable carrier or excipient.

A peptide of the invention may be used for methods for immunizing or treating humans and/or animals with the disease caused by infection with E. faecalis. Therefore, the peptide may be used within a pharmaceutical composition. The pharmaceutical composition of the present invention may further encompass pharmaceutically acceptable carriers and/or excipients. The pharmaceutically acceptable carriers and/or excipients useful in this invention are conventional and may include buffers, stabilizers, diluents, preservatives, and solubilizers. Remington's Pharmaceutical Sciences, by E. W. Martin, Mack Publishing Co., Easton, Pa., 15th Edition (1975), describes compositions and formulations suitable for pharmaceutical delivery of the (poly)peptides herein disclosed. In general, the nature of the carrier or excipients will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g. powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In a preferred embodiment the pharmaceutical composition further comprises an immunostimulatory substance such as an adjuvant. The adjuvant can be selected based on the method of administration and may include mineral oil-based adjuvants such as Freund's complete and incomplete adjuvant, Montanide incomplete Seppic adjuvant such as ISA, oil in water emulsion adjuvants such as the Ribi adjuvant system, syntax adjuvant formulation containing muramyl dipeptide, or aluminum salt adjuvants. Preferably, the adjuvant is a mineral oil-based adjuvant.

In a more preferred embodiment the immunostimulatory substance is selected from the group comprising polycationic polymers, especially polycationic peptides, immunostimulatory oligodeoxynucleotides (ODNs), especially Oligo(dIdC)₁₃ peptides containing at least two LysLeuLys motifs, especially KLKLLLLLKLK, neuroactive compounds, especially human growth hormone, alum, Freund's complete or incomplete adjuvants, or combinations thereof.

In a still more preferred embodiment the immunostimulatory substance is a combination of either a polycationic polymer and immunostimulatory oligodeoxynucleotides or of a peptide containing at least two LysLeuLys motifs and immunostimulatory oligodeoxynucleotides, preferably a combination of KLKLLLLLKLK and Oligo(dIdC)₁₃.

The term “Oligo(dIdC)₁₃” as used in the present invention means a phosphodiester backboned single-stranded DNA molecule containing 13 deoxy (inosine-cytosine) motifs, also de fined by the term [oligo-d(IC)₁₃]. The exact sequence is 5′-dIdCdIdCdIdCdIdCdIdCdIdCdIdCdIdCdIdCdIdCdIdCdIdCdIdC-3′. Oligo(dIdC)₁₃ can also be defined by the terms (oligo-dIC₂₆); oligo-dIC₂₆₋ₘₑᵣ; oligo-deoxy IC, 26-mer; or oligo-dIC, 26-mer, as specified for example in WO 01/93903 and WO 01/93905.

In an even more preferred embodiment of the pharmaceutical composition of the present invention the polycationic polymer is a polycationic peptide, especially polyarginine.

In an even more preferred embodiment of the invention the immunostimulatory substance is at least one immunostimulatory nucleic acid. Immunostimulatory nucleic acids are e.g. neutral or artificial CpG containing nucleic acids, short stretches of nucleic acids derived from non-vertebrates or in form of short oligonucleotides (ODNs) containing non-methylated cytosine-guanine dinucleotides (CpG) in a defined base context (e.g. as described in WO 96/02555). Alternatively, also nucleic acids based on inosine and cytidine as e.g. described in WO 01/93903, or deoxynucleic acids containing deoxy-inosine and/or deoxyuridine residues (described in WO 01/93905 and WO 02/095027) may preferably be used as immunostimulatory nucleic acids in the present invention. Preferably, mixtures of different immunostimulatory nucleic acids are used in the present invention. Additionally, the aforementioned polycationic compounds may be combined with any of the immunostimulatory nucleic acids as aforementioned. Preferably, such combinations are according to the ones described in WO 01/93905, WO 02/32451, WO 01/54720, WO 01/93903, WO 02/13857, WO 02/095027 and WO 03/047602.

In addition or alternatively, the pharmaceutical composition may comprise a neuroactive compound. Preferably, the neuroactive compound is human growth factor, e.g. described in WO 01/24822. Also preferably, the neuroactive compound is combined with any of the polycationic compounds and/or immunostimulatory nucleic acids as defined above.

The pharmaceutical composition encompasses at least one peptide of the invention; however, it may also contain a cocktail (i.e., a simple mixture) comprising different peptides (including fragments and other variants) of the invention, optionally mixed with different antigenic proteins or peptides of other pathogens. Such mixtures of these peptides, polypeptides, proteins or fragments or variants thereof are useful e.g. in the generation of desired antibodies to a wide spectrum of enterococci isolates. The peptide(s) of the present invention may also be used in the form of a pharmaceutically acceptable salt. Suitable acids and bases which are capable of forming salts with the peptides of the present invention are well known to those of skill in the art, and include inorganic and organic acids and bases.

Still another subject of the invention is a pharmaceutical composition containing a nucleic acid selected from the group consisting of:

(i) a nucleic acid of the invention and/or a nucleic acid complementary thereto, and
(ii) optionally a pharmaceutically acceptable carrier or excipient.

In another embodiment, the nucleic acid sequences of this invention, alone or in combination with nucleic acid sequences encoding other antigens or antibodies from other pathogenic microorganisms, may further be used as components of a pharmaceutical composition. The composition may be used for immunizing or treating humans and/or animals with the disease caused by infection with E. faecalis. The pharmaceutically acceptable carrier or excipient may be as defined above.

For use in the preparation of the therapeutic or vaccine compositions, nucleic acid delivery compositions and methods are useful, which are known to those of skill in the art. The nucleic acid of the invention may be employed in the methods of this invention or in the compositions described herein as DNA sequences, either administered as naked DNA, or associated with a pharmaceutically acceptable carrier and provide for in vivo expression of the antigen, peptide or polypeptide. So-called “naked DNA” may be used to express the antigen, peptide or polypeptide of the invention in vivo in a patient. (See, e.g., J. Cohen, Science, 259:1691-1692, which describes similar uses of “naked DNA”). For example, “naked DNA” associated with regulatory sequences may be administered therapeutically or as part of the vaccine composition e.g., by injection.

Alternatively, a nucleic acid encoding the antigens or peptides of the invention or a nucleic acid complementary thereto may be used within a pharmaceutical composition, e.g. in order to express the antigens or peptides or polypeptides of the invention in vivo, e.g., to induce antibodies.

A preferred embodiment of the invention relates to a pharmaceutical composition, wherein the nucleic acid according to the invention is comprised in a vector and/or a cell. Vectors and cells suitable in the context of the present invention are described above. Vectors are particularly employed for a DNA vaccine. An appropriate vector for delivery may be readily selected by one of skill in the art. Exemplary vectors for in vivo gene delivery are readily available from a variety of academic and commercial sources, and include, e.g., adeno-associated virus (International patent application No. PCT/US91/03440), adenovirus vectors (M. Kay et al., Proc. Natl. Acad. Sci. USA, 91:2353 (1994); S. Ishibashi et al., J. Clin. Invest., 92:883 (1993)), or other viral vectors, e.g., various poxviruses, vaccinia, etc. Recombinant viral vectors, such as retroviruses or adenoviruses, are preferred for integrating the exogenous DNA into the chromosome of the cell.

In order to be able to overcome the diseases, a vaccination may not be suitable or not possible due to the advanced and/or fast disease progression. Thus it would be very beneficial to provide monoclonal or polyclonal antibody therapies which target antigenic proteins of E. faecalis and have the potential to support a therapy of an infection or eliminate the pathogen and the disease altogether.

Therefore, another subject of the invention relates to an antibody or functional active fragment thereof which binds specifically to the antigen of the invention.

In a preferred embodiment the antibody is a monoclonal, polyclonal, chimeric or humanized antibody or functional active variant thereof. In another preferred embodiment the functional active fragment comprises a Fab fragment.

Antibodies generated against the antigens, fragments or variants thereof of the present invention can be obtained by direct injection of the antigens, fragments or variants thereof into an animal or by administering the antigens, fragments or variants thereof to an animal, preferably a non-human. The antibody so obtained will then bind the antigens, fragments or variants. Such antibodies can then be used to isolate reactive antigens, fragments or variants thereof from tissue expressing those.

For preparation of monoclonal antibodies, any technique known in the art, which provides antibodies produced by continuous cell line cultures, e.g. a hybridoma cell line, can be used.

Techniques described for the production of single chain antibodies (U.S. Pat. No. 4,946,778) can be adapted to produce single chain antibodies to the antigens, fragments or variants thereof according to this invention. Also, transgenic mice or other organisms such as other mammals may be used to express humanized antibodies to antigens, fragments or variants thereof according to this invention.

Still another subject of the invention relates to a hybridoma cell line which produces the antibody of the invention.

Hybridoma cell lines expressing desirable monoclonal antibodies are generated by well-known conventional techniques. The hybridoma cell can be generated by fusing a normal-activated, antibody-producing B cell with a myeloma cell. In the context of the present invention the hybridoma cell is able to produce an antibody specifically binding to the antigen of the invention.

Similarly, desirable high titer antibodies are generated by applying known recombinant techniques to the monoclonal or polyclonal antibodies developed to these antigens (see, e.g., PCT Patent Application No. PCT/GB85/00392; British Patent Application Publication No. GB2188638A; Amit et al., Science, 233:747-753 (1986); Queen et al., Proc. Natl. Acad. Sci. USA, 86:10029-10033 (1989); WO 90/07861; Riechmann et al., Nature, 332:323-327 (1988); Huse et al., Science, 246:1275-1281 (1988)).

The present invention also provides a method for producing an antibody according to the invention, characterized by the following steps:
(a) administering an effective amount of the peptide according to the invention to an animal; and
(b) isolating the antibody produced by the animal in response to the administration of step (a) from the animal.

Another subject of the invention relates to a method for producing an antibody according to the invention, characterized by the following steps:
(a) contacting a B cell with an effective amount of the peptide according to the invention;
(b) fusing the B cell of step (a) with a myeloma cell to obtain a hybridoma cell; and
(c) isolating the antibody produced by the cultivated hybridoma cell.

More particularly, the antibody may be produced by initiating an immune response in a non-human animal by administrating a peptide of the invention to an animal, removing an antibody containing body fluid from said animal, and producing the antibody by subjecting said antibody containing body fluid to further purification steps. Alternatively, the antibody may be produced by initiating an immune response in a non-human animal by administrating an antigen, fragment or variant thereof, as defined in the present invention, to said animal, removing the spleen or spleen cells from said animal and/or producing hybridoma cells of said spleen or spleen cells, selecting and cloning hybridoma cells specific for said antigen, fragment or variant thereof and producing the antibody by cultivation of said cloned hybridoma cells.

In a preferred embodiment the antibody produced according to a method of the invention is additionally purified. Methods of purification are known to the skilled artisan.

The antibody may be used in methods for treating an infection. Accordingly, still another subject of the invention relates to a pharmaceutical composition comprising the antibody of the invention. The pharmaceutical composition may encompass further components as detailed above. The composition may further encompass substances increasing their capacity to stimulate T cells. These include T helper cell epitopes, lipids or liposomes or preferred modifications as described in WO 01/78767. Another way to increase the T cell stimulating capacity of epitopes is their formulation with immune stimulating substances for instance cytokines or chemokines like interleukin-2, -7, -12, -18, class I and II interferons (IFN), especially IFN-gamma, GM-CSF, TNF-alpha, flt3-ligand and others.

Still another subject of the invention is a pharmaceutical composition, especially a vaccine, comprising the protective peptide or the nucleic acid or the antibody or functional fragments thereof as defined by the invention, useful for the immunization of a subject against an infection or the treatment of a subject having an infection, wherein the infection is preferably caused by E. faecalis.

In another aspect of the invention the peptide or the nucleic acid of the invention is used for the manufacture of a medicament for the immunization or treatment of a subject, preferably against E. faecalis. Alternatively, the peptide or the nucleic acid of the invention is used in a method of immunizing or treating a subject in need thereof, wherein an effective amount of the peptide or the nucleic acid of the invention is administered to the subject. The subject may be immunized in order to prevent an infection, particularly an E. faecalis infection, or may be treated to ameliorate or cure an infection, particularly an E. faecalis infection. The determination of the effective amount to be administered is within the knowledge of the skilled practitioner. Exemplary amounts are mentioned below.

The peptides or the nucleic acids of the invention are generally useful for inducing an immune response in a subject. The pharmaceutical composition (vaccine) used for immunization may be administered to a subject susceptible to infection by E. faecalis, preferably mammals, and still more preferably humans, in any conventional manner, including oral, intranasal, intramuscular, intra-lymph node, intradermal, intraperitoneal, subcutaneous, and combinations thereof, but most preferably through subcutaneous or intramuscular injection. The volume of the dose when subcutaneous or intramuscular injection is the selected administration route is preferably up to about 5 ml, still more preferably between 0.3 ml and 3 ml, and most preferably about 0.5 to 1 ml. The amount of protein comprising the antigen in each dose should be enough to confer effective immunity against and decrease the risk of developing clinical signs resulting from E. faecalis infection to a subject receiving a vaccination therewith. Preferably, the unit dose of protein should be up to about 5 μg protein/kg body weight, more preferably between about 0.2 to 3 μg, still more preferably between about 0.3 to 1.5 μg, more preferably between about 0.4 to 0.8 μg, and still more preferably about 0.6 μg. Alternative preferred unit doses of protein could be up to about 6 μg protein/kg body weight, more preferably between about 0.05 to 5 μg, still more preferably between about 0.1 to 4 μg. The dose is preferably administered 1 to 3 times, e.g. with an interval of 1 to 3 weeks. Preferred amounts of protein per dose are from approximately 1 μg to approximately 1 mg, more preferably from approximately 5 μg to approximately 500 μg, still more preferably from approximately 10 μg to approximately 250 μg and most preferably from approximately 25 μg to approximately 100 μg.

In still another aspect of the invention the antibody of the invention or functional fragment thereof is used for the manufacture of a medicament for the treatment of an infection, preferably an E. faecalis infection. Alternatively, the antibody of the invention is used in a method of treating a subject in need thereof, wherein an effective amount of the antibody of the invention is administered to the subject. The subject may be treated to ameliorate or cure an infection, particularly an E. faecalis infection. The determination of the effective amount to be administered is within the knowledge of the skilled practitioner. Exemplary amounts are mentioned below.

The treatment involves administering an effective amount of an antibody of the invention to a subject, preferably a mammal, more preferably a human. Thus, antibodies against the antigens, fragments or variants thereof of the present invention may be employed to inhibit and/or treat infections, particularly bacterial infections and especially infections arising from E. faecalis.

An “effective amount” of a peptide, nucleic acid or an antibody of the invention may be calculated as that amount capable of exhibiting an in vivo effect, e.g. preventing or ameliorating a sign or symptom of infection, particularly E. faecalis infection. Such amounts may be determined by one of skill in the art. Preferably, such a composition is administered parenterally, preferably intramuscularly or subcutaneously. However, it may also be formulated to be administered by any other suitable route, including orally or topically. The selection of the route of delivery and dosage of such therapeutic compositions is within the skill of the art.

Treatment in the context of the present invention refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

Another subject of the invention relates to a method of diagnosing an E. faecalis infection comprising the steps of:
(a) contacting a sample obtained from a subject with the peptide according to the invention; and
(b) detecting the presence of an antibody against E. faecalis in the sample.

The peptides of the invention may be used for the detection of the E. faecalis. Preferably such detection is for diagnosis, more preferable for the diagnosis of a disease, most preferably for the diagnosis of an E. faecalis infection. The peptides or polypeptides may be used to detect the presence of an E. faecalis-specific antibody or fragment thereof e.g. in a sample obtained from a subject. The sample may be e.g. a blood sample. Alternatively, the presence of an E. faecalis-specific antigen can be detected using an antibody of the invention.

Accordingly, an alternative method of diagnosing an E. faecalis infection comprises the steps of:
(a) contacting a sample obtained from a subject with the antibody according to the invention; and
(b) detecting the presence of an antigen of E. faecalis in the sample.

The present invention also relates to diagnostic assays such as quantitative and diagnostic assays for detecting levels of the peptides or antibodies of the present invention in cells and tissues or body fluids, including determination of normal and abnormal levels. Assay techniques that can be used to determine levels of a peptide or an antibody, in a sample derived from a host are well known to those of skill in the art. Such assay methods include radioimmunoassays, competitive-binding assays, Western Blot analysis and ELISA assays (Enzyme-linked Immunosorbent Assays). Among these, ELISAs frequently are preferred. An ELISA assay initially comprises preparing an antibody specific to the peptide, particularly the antigen, preferably a monoclonal antibody. In addition, a reporter antibody generally is prepared which binds to the monoclonal antibody. The reporter antibody is attached to a detectable reagent such as radioactive, fluorescent or enzymatic reagent, such as horseradish peroxidase enzyme.

The peptides or antibodies of the present invention may also be used for the purpose of or in connection with an array. More particularly, at least one of the peptides or antibodies of the present invention may be immobilized on a support. Said support typically comprises a variety of antigens and fragments thereof whereby the variety may be created by using one or several of the peptides or antibodies of the present invention. The characterizing feature of such array as well as of any array in general is the fact that at a distinct or predefined region or position on said support or a surface thereof, a distinct polypeptide is immobilized. Because of this any activity at a distinct position or region of an array can be correlated with a specific polypeptide. The number of different peptides or antibodies of the present invention immobilized on a support may range from as little as 10 to several 1000 different peptides or antibodies of the present invention.

The manufacture of such arrays is known to the one skilled in the art and, for example, described in U.S. Pat. No. 5,744,309. The array preferably comprises a planar, porous or non-porous solid support having at least a first surface. Preferred support materials are, among others, glass or cellulose. It is also within the present invention that the array is used for any of the diagnostic applications described herein. Apart from the peptides or antibodies of the present invention also the nucleic acid molecules according to the present invention may be used for the generation of an array as described above.

In a last aspect, the peptide according to the invention is used for the isolation and/or purification and/or identification of an interaction partner of the peptide (a ligand), wherein the isolation and/or purification and/or identification of the ligand may be carried out as detailed below or as known to the person skilled in the art. In a preferred embodiment of the invention an affinity device may be used. The affinity device may comprise as least a support material and any peptide according to the present invention, which is attached to the support material. Because of the specificity of the peptides according to the present invention for their target cells or target molecules or their interaction partners, the peptides allow a selective removal of their interaction partner(s) from any kind of sample applied to the support material provided that the conditions for binding are met. The sample may be a biological or medical sample, including but not limited to, fermentation broth, cell debris, cell preparation, tissue preparation, organ preparation, blood, urine, lymph liquid, liquor and the like. The peptide may be attached to the matrix in a covalent or non-covalent manner. Suitable support material is known to the one skilled in the art and can be selected from the group comprising cellulose, silicon, glass, aluminum, paramagnetic beads, starch and dextrane.

More particularly, the isolation and/or purification and/or identification of the ligand may be carried out by contacting an isolated or immobilized peptide according to the invention with a candidate ligand under conditions to permit binding of the candidate ligand to the peptide, wherein the test system comprises a component capable of providing a detectable signal in response to the binding of the candidate ligand to said peptide; and detecting the presence or absence of a signal generated in response to the binding of the ligand to the peptide. The ligand may be an agonist or an antagonist.

Test systems for detection binding of a ligand are known to the skilled artisan and include e.g. binding assays with labeled ligand such as radioligands, fluorescence-labeled ligands or enzyme-labeled ligands.

The test compound can be any test compound either naturally occurring or chemically synthesized. Naturally occurring test compounds include in particular antibodies, preferably those showing similarity to the antibodies of the invention. In one preferred embodiment of the invention the test compound is provided in the form of a chemical compound library. Chemical compound libraries include a plurality of chemical compounds and have been assembled from any of multiple sources, including chemically synthesized molecules and natural products, or have been generated by combinatorial chemistry techniques. They are especially suitable for high throughput screening. They may be comprised of chemical compounds of a particular structure or compounds of a particular creature such as a plant.

The ligands identified may be employed, for instance, to inhibit diseases arising from infection with a member of the Enterococcus genus (Enterococci), especially E. faecalis and may therefore be formulated in a pharmaceutical composition.

The present invention is further illustrated by the following figures, examples and the sequence listing, from which further features, embodiments and advantages may be taken. It is to be understood that the present examples are given by way of illustration only and not by way of limitation of the disclosure.

FIGS. 1 to 21 demonstrate the protection achieved by active immunization with selected E. faecalis antigens in a rat model of catheter-induced endocarditis.

Table 1 shows the peptides of the present invention to be related to proteins from WO 04/106367.

Table 2 shows the DNA constructs made for expression of recombinant E. faecalis proteins prepared for assessing protection.

Table 3 shows the range of protection conferred by E. faecalis recombinant proteins in a rat model of catheter-induced endocarditis.

Table 4 presents all 46 E. faecalis strains utilized for PCR and sequencing of vaccine-antigen encoding ORFs.

Table 5 shows a summary on the sequence conservation of the selected 9 E. faecalis genes in forty-six isolates.

### FIGURES

FIGS. 1 to 21. Protection Achieved by Active Immunization with the E. Faecalis Antigens in a Rat Model of Catheter-Induced Endocarditis.

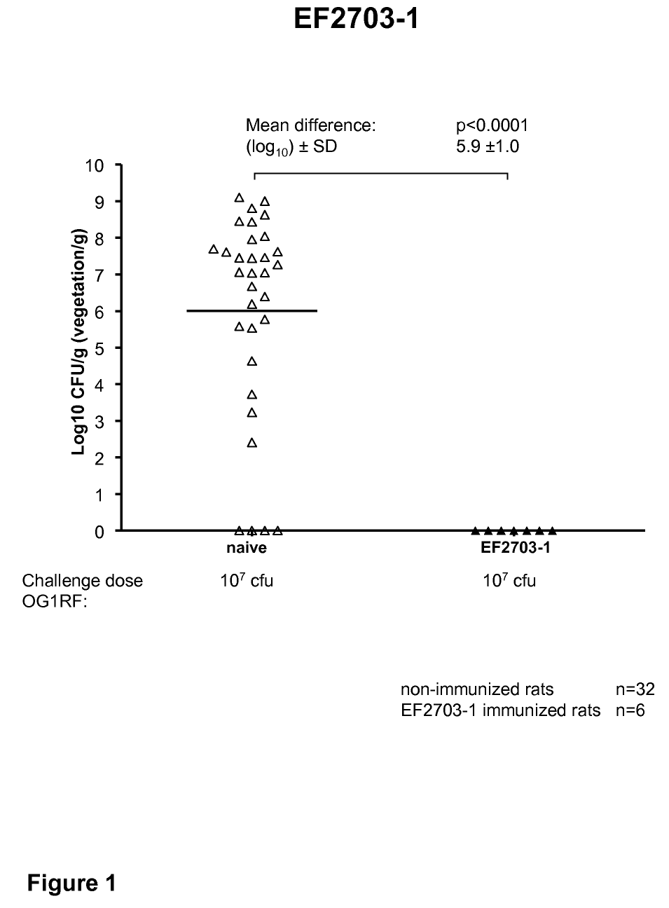

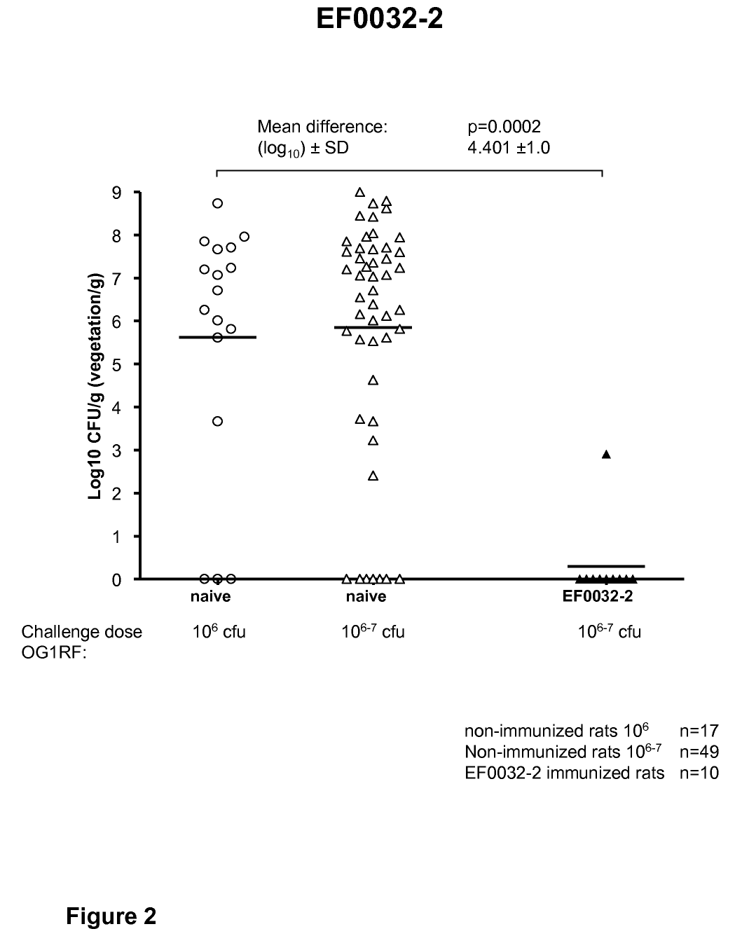

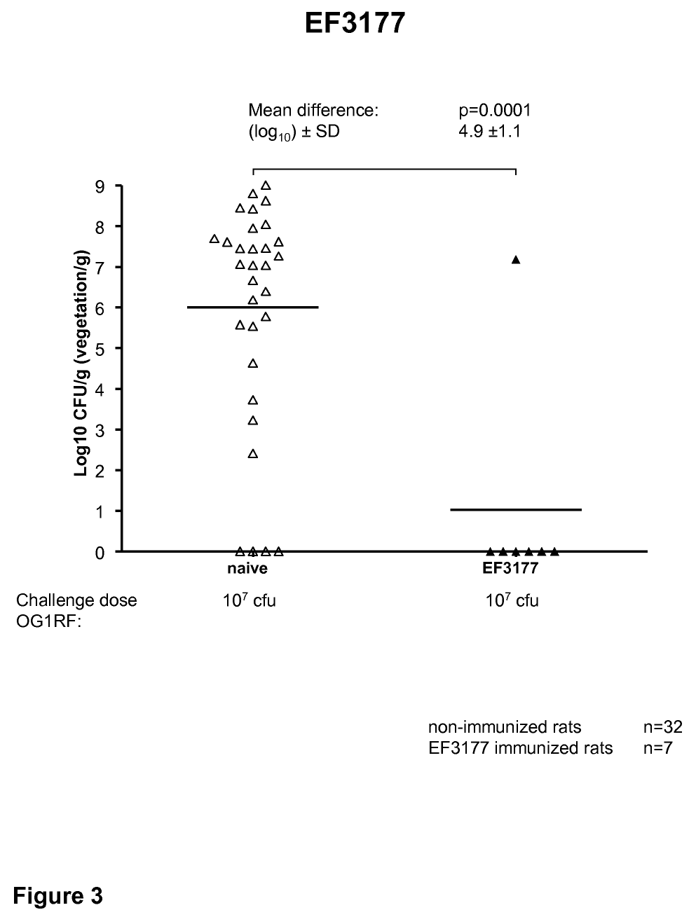

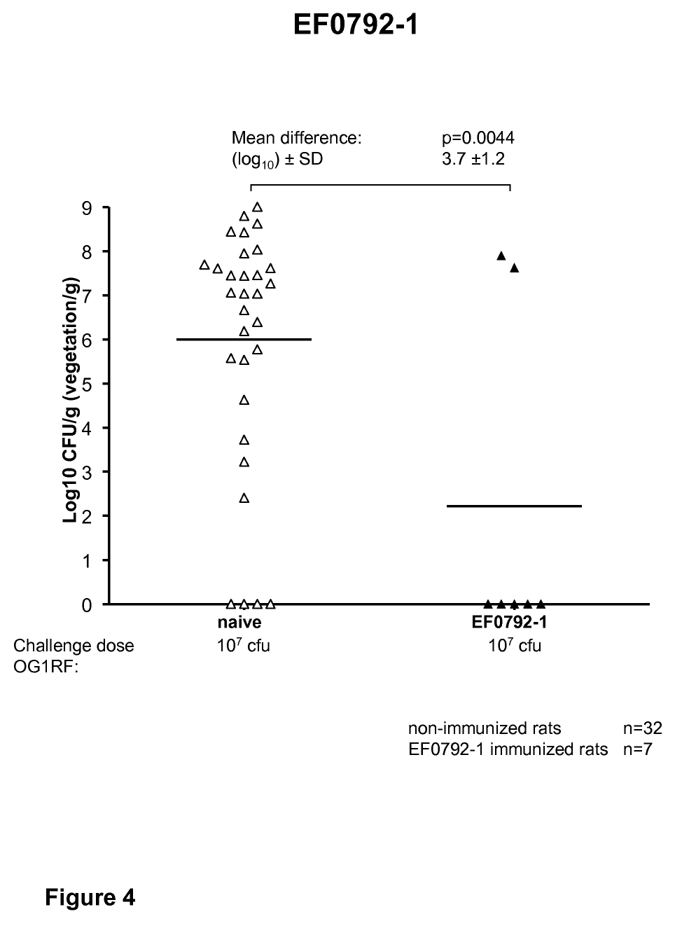

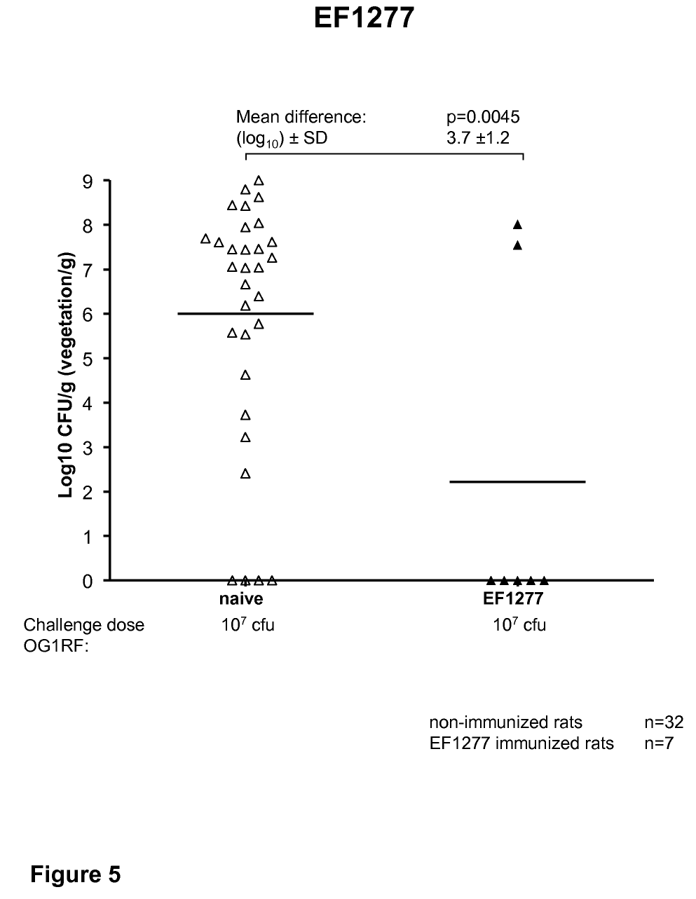

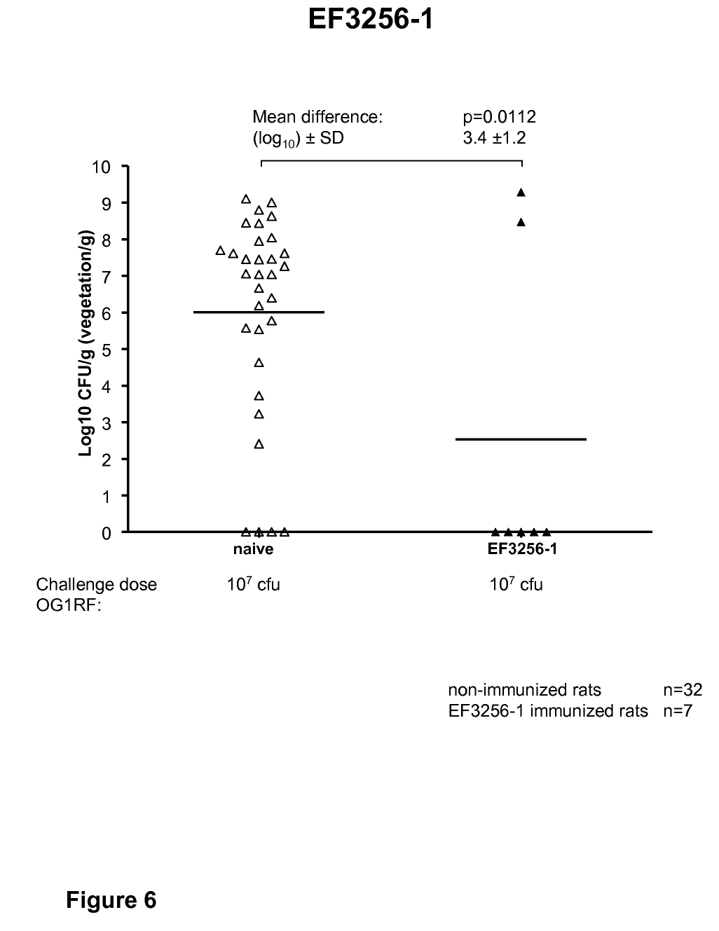

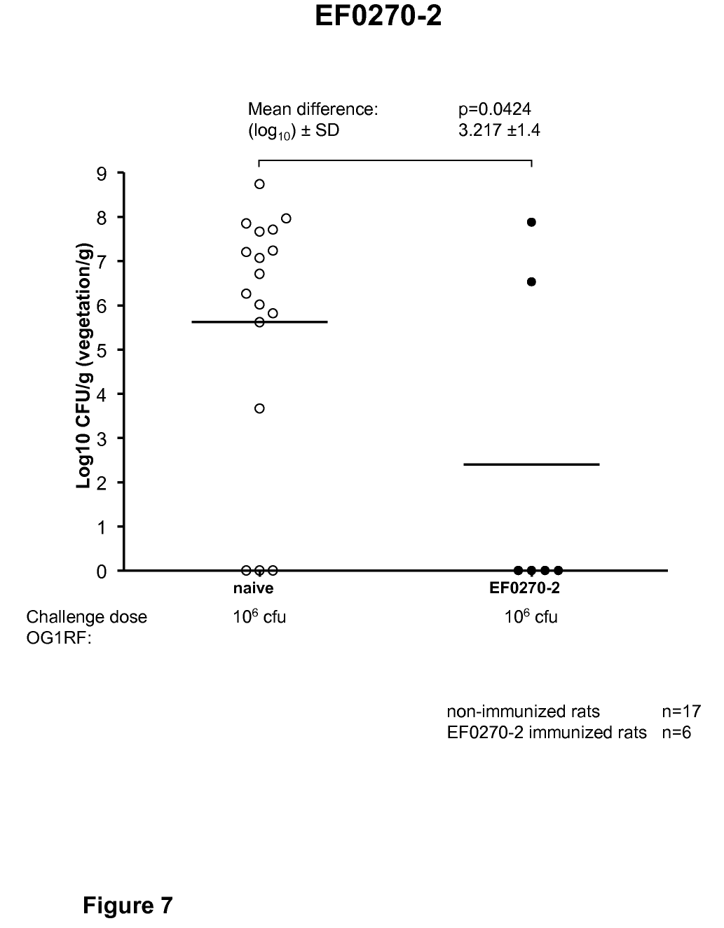

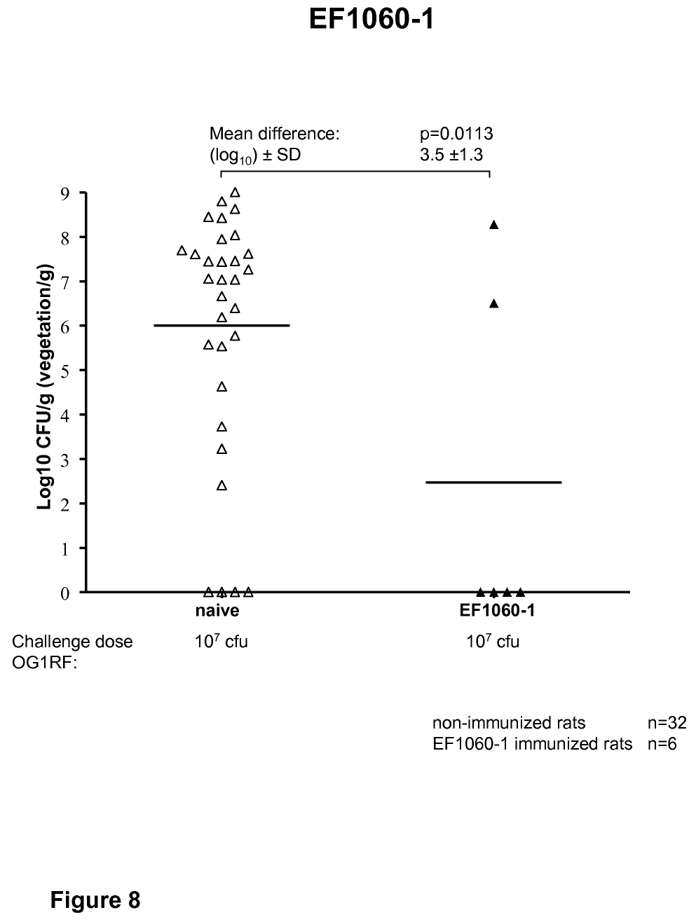

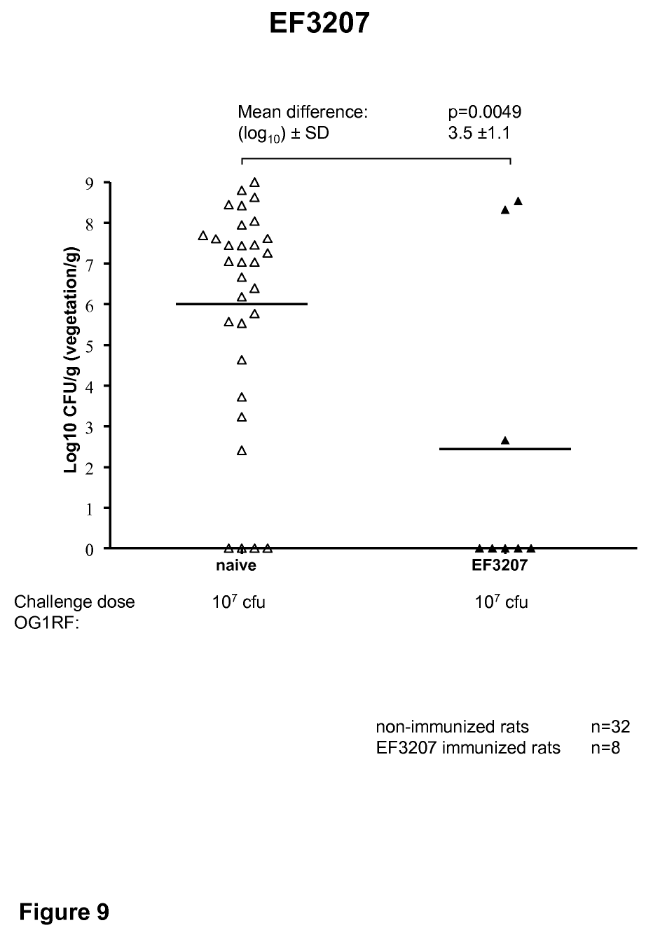

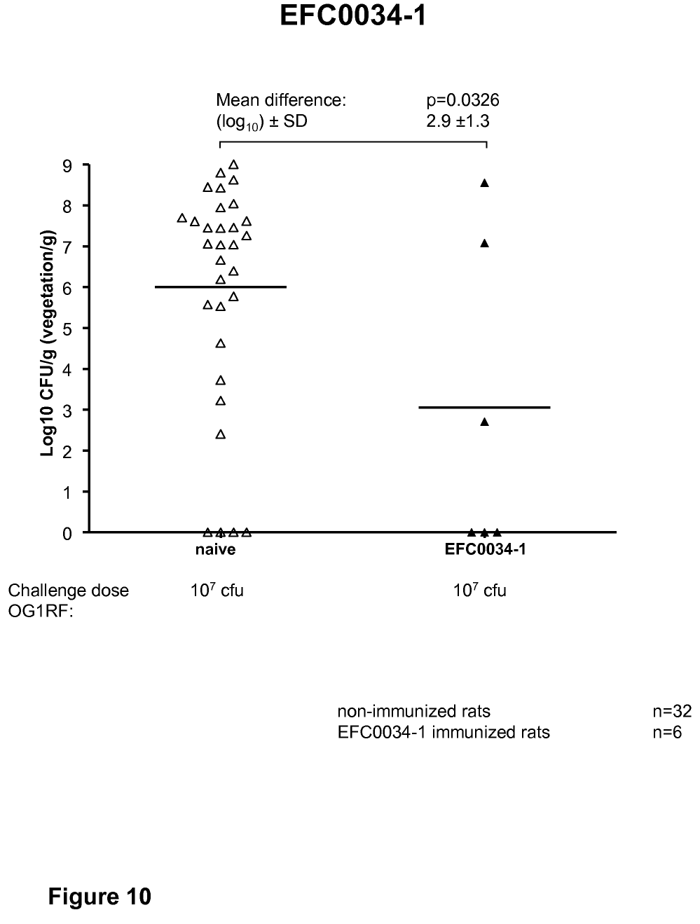

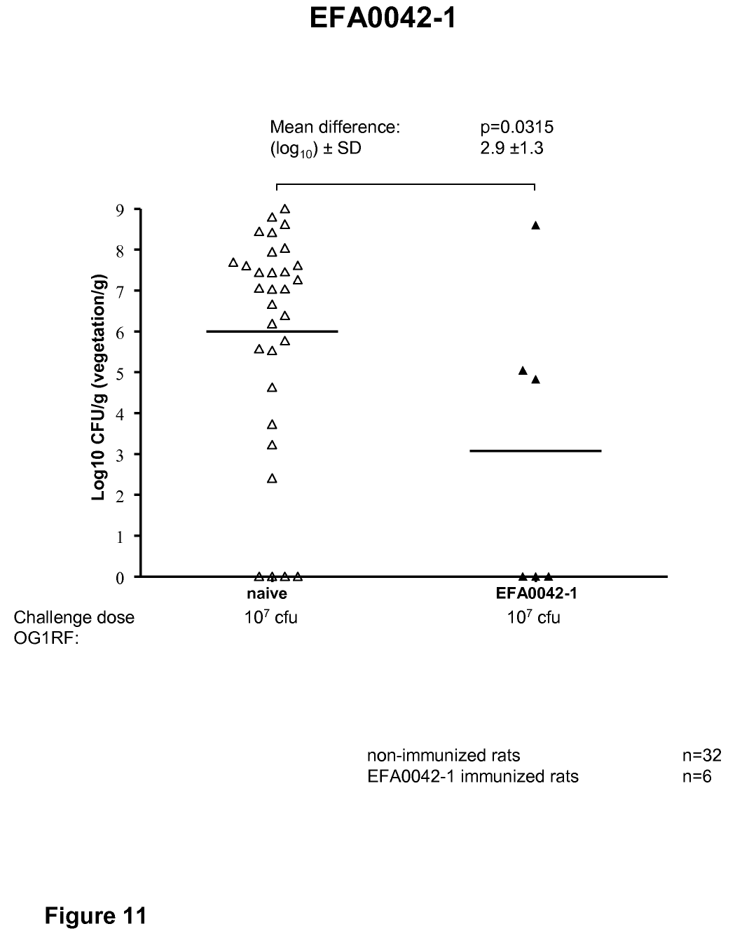

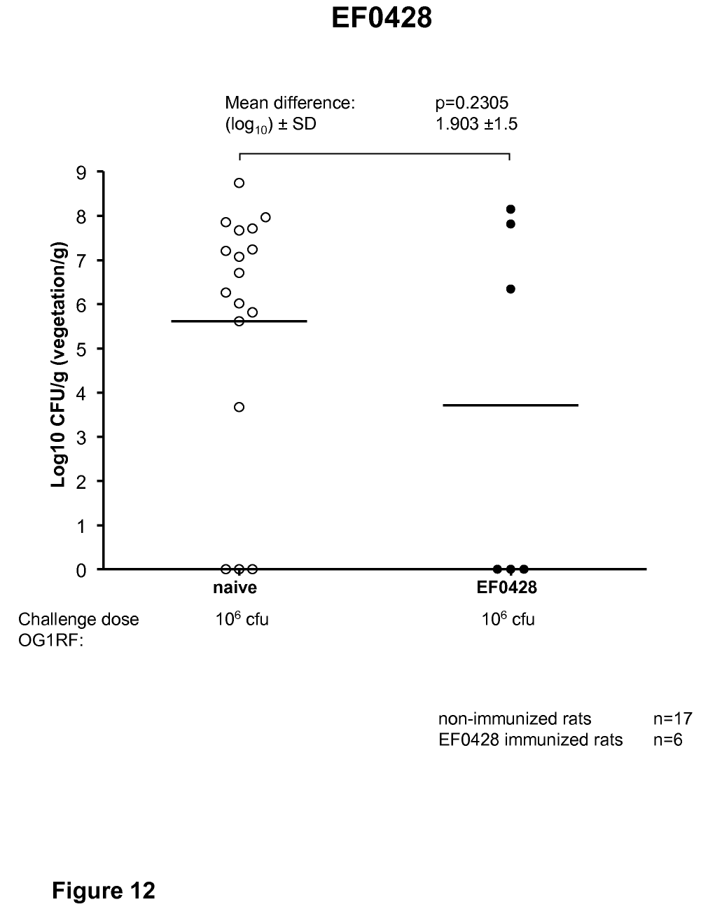

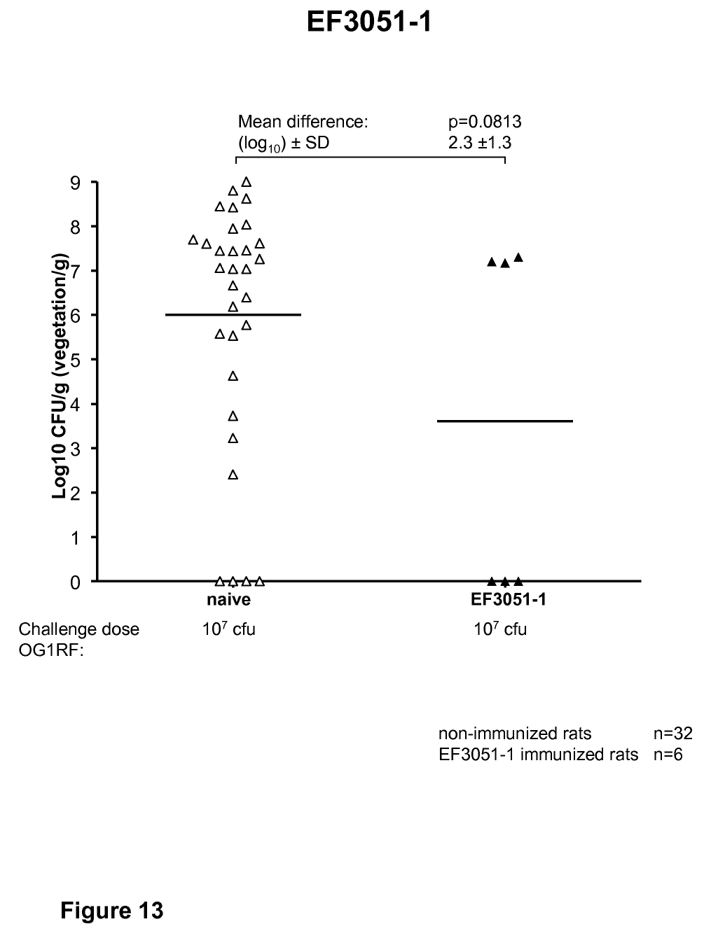

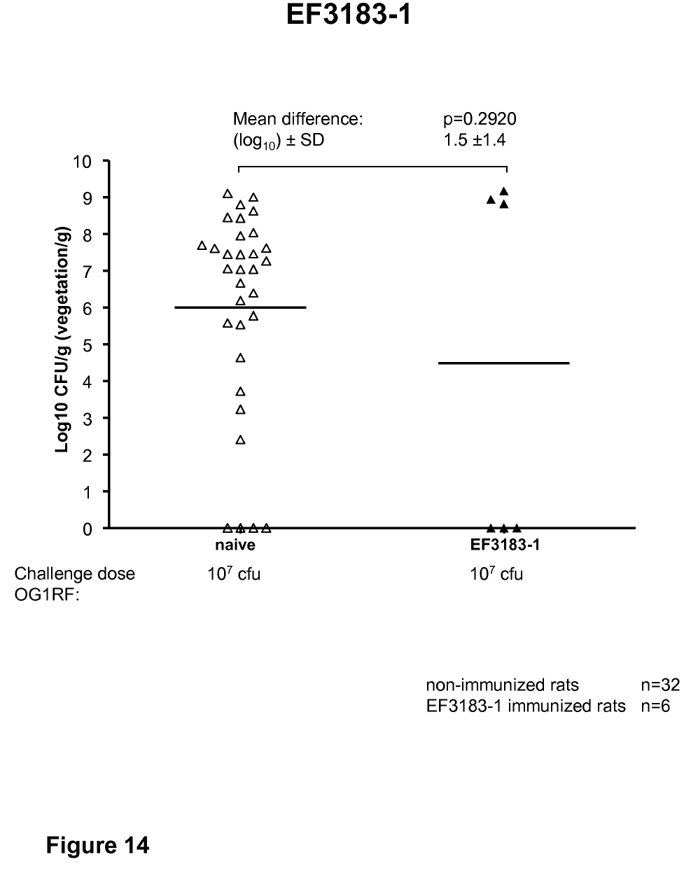

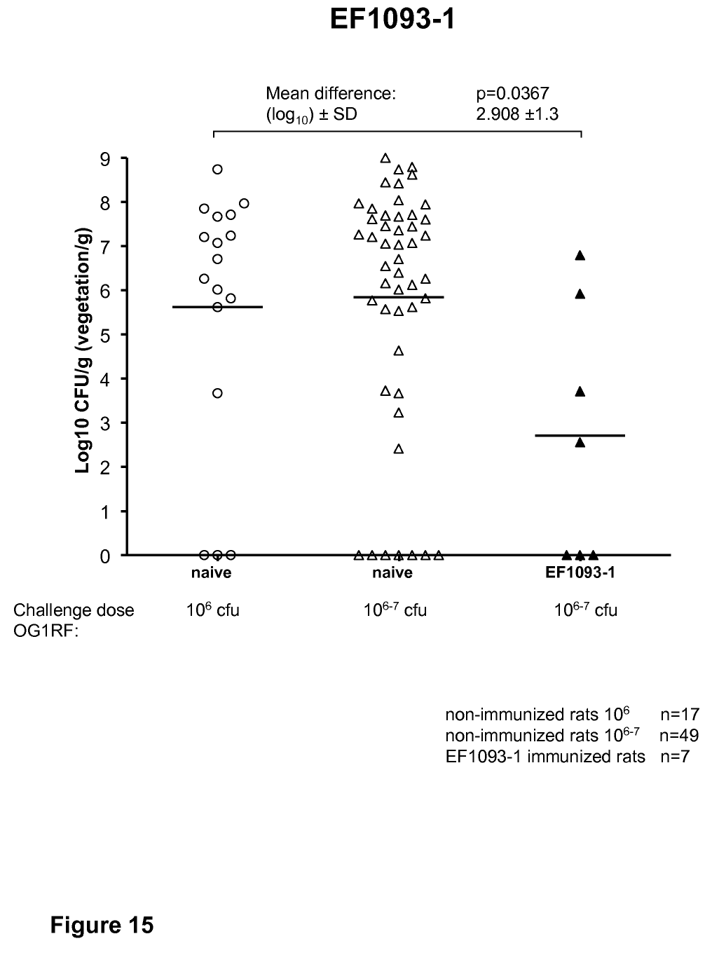

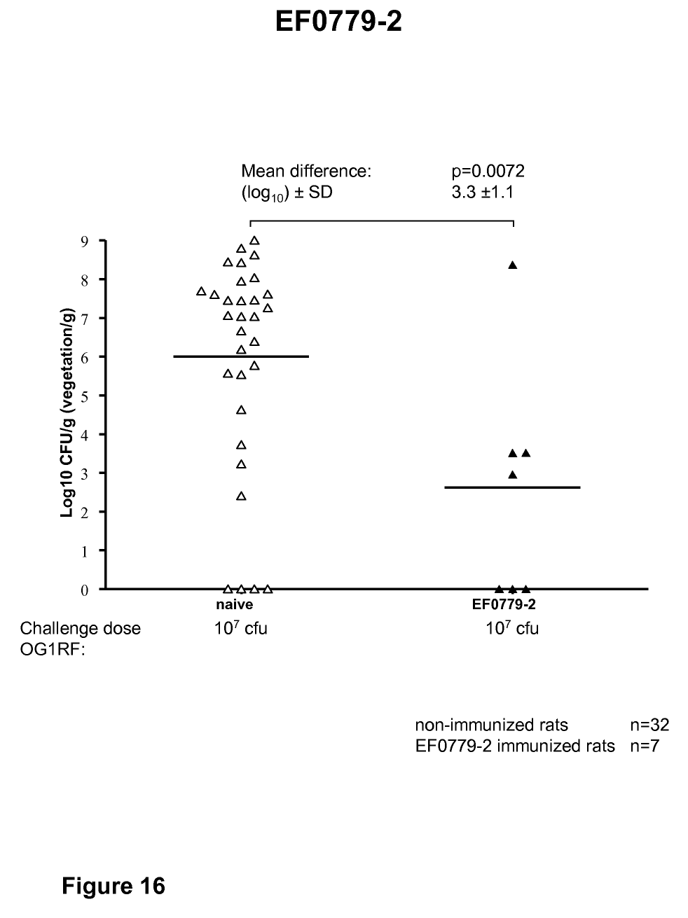

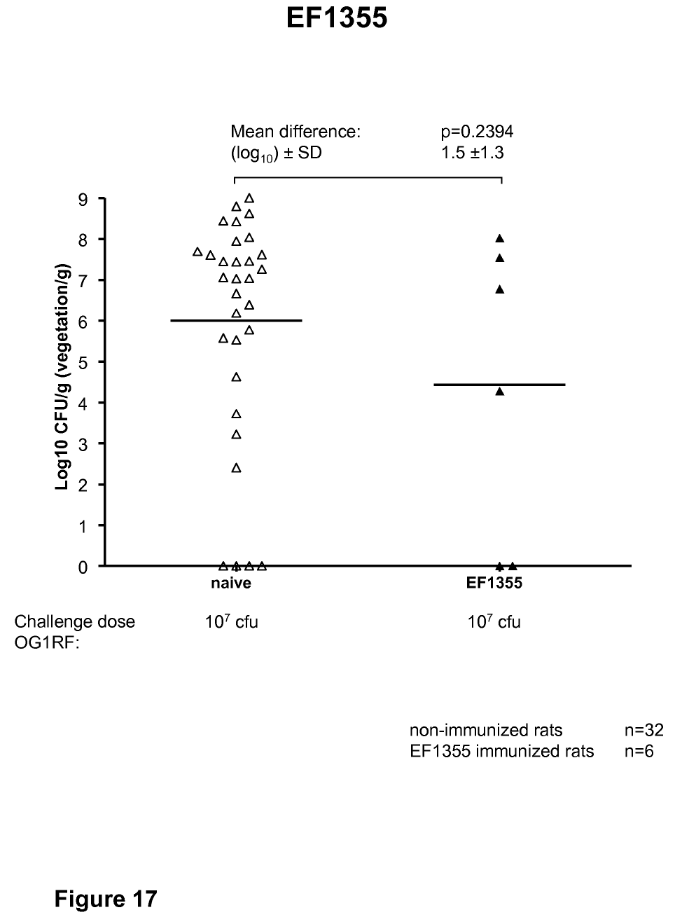

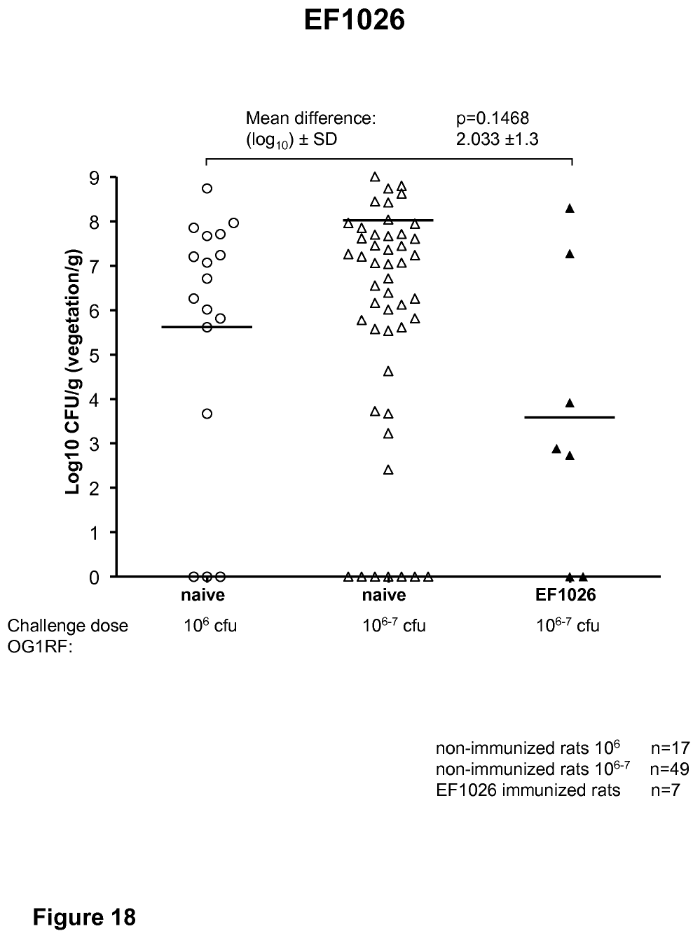

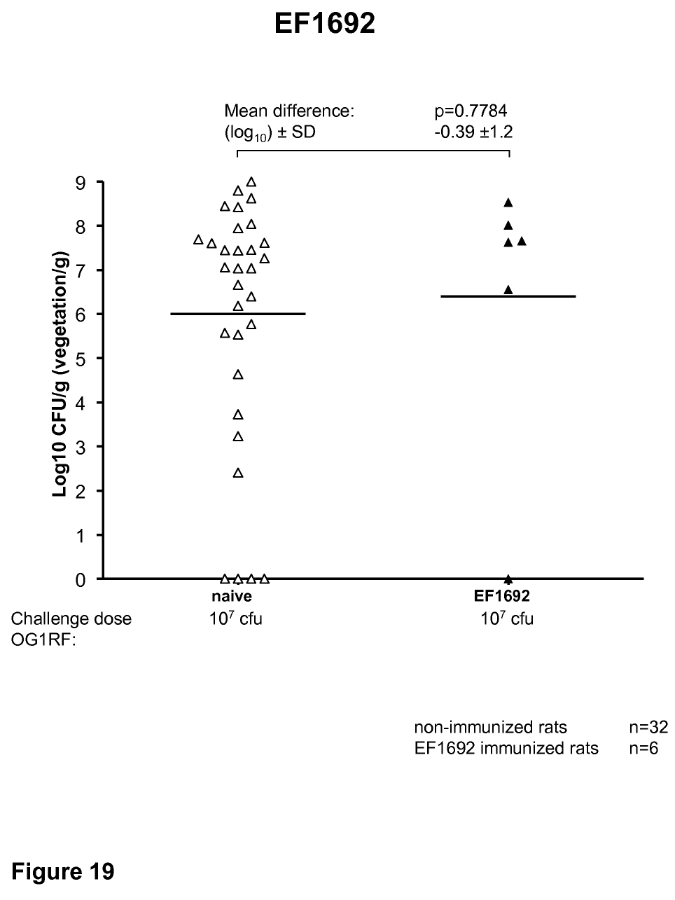

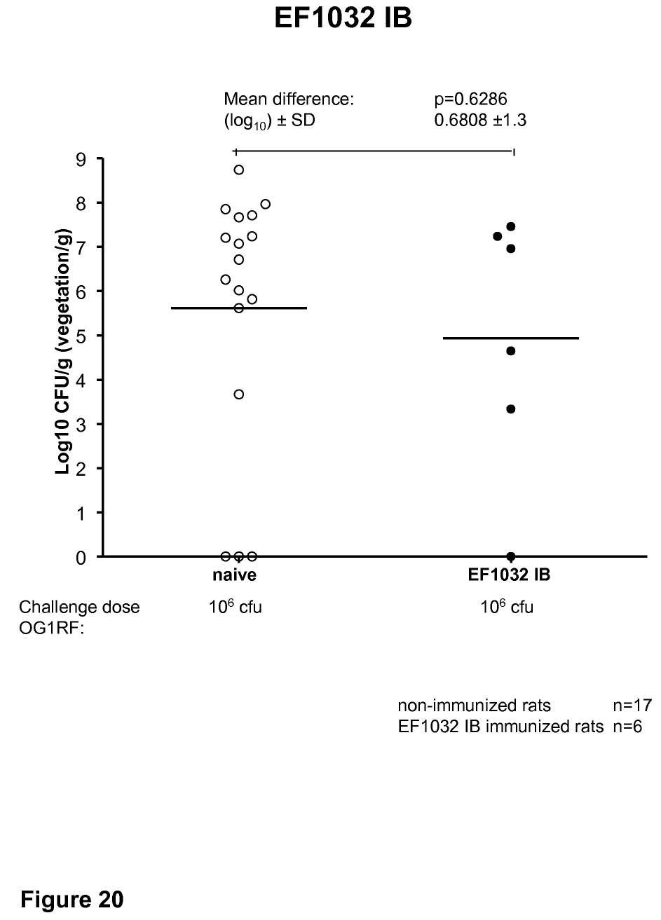

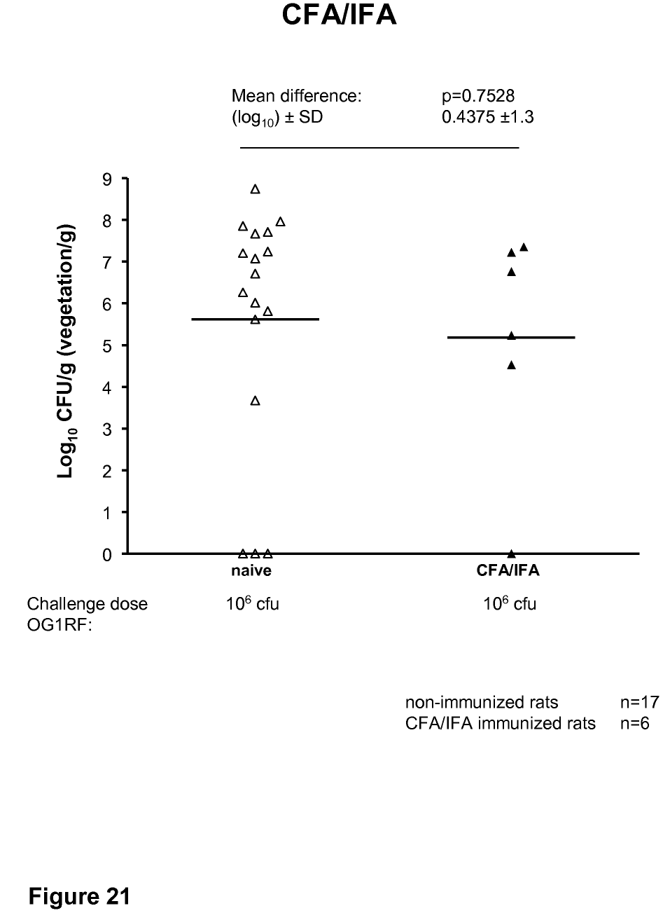

Male Sprague-Dawley rats immunized with recombinant E. faecalis proteins (each figure represents the data from one recombinant protein) were subsequently challenged i.v. with 1×10⁶-10⁷ CFU of E. faecalis strain OG1RF for induction of endocarditis. Enterococcus faecalis OG1RF in the vegetations of control rats versus OG1RF in the vegetations of recombinant protein immunized rats are depicted (significance determined by the unpaired t-test). Data are expressed as log 10 colony-forming units per gram recovered from vegetation homogenates ˜48 h post infection; the log 10 no. of colony-forming units per gram for vegetation were combined and averaged. Horizontal bars represent geometric means. Circles represent experiments where the challenge dose was 10⁶ cfu, and triangles represent challenge doses of 10⁷ cfu, or data where challenge dose of 10⁶ and 10⁷ were combined.

### EXAMPLES

### Example 1

### Identification of Enterococcus faecalis Antigens Eliciting Protective Immune Responses in a Rat Model of Catheter-Induced E. faecalis Endocarditis

### Experimental Procedures

### Cloning and Expression of Recombinant Enterococcal Proteins

### Cloning of Genes/DNA Fragments:

The gene/DNA fragment of interest was amplified from genomic DNA of E. faecalis (strain V583; serogroup CPS-C) by PCR using gene specific primers. Apart from the gene specific part, the primers had restriction sites that aided in a directional cloning of the amplified PCR product. The gene annealing (specific) part of the primer ranged between 15-30 bases in length. The PCR products obtained were digested with the appropriate restriction enzymes and cloned into the pET28b (+) or pET41b (+) vector (Novagen) for His-tagged proteins. The constructs are listed in Table 2. Once the recombinant plasmid was confirmed to contain the gene of interest, E. coli BL21 Star™ (DE3) cells (Invitrogen) that served as expression host were transformed.

### Expression and Purification of Proteins:

E. coli BL21 Star™ cells harboring the recombinant plasmid were grown into log phase in the required culture volume. Once an OD₆₀₀ₙₘ of 0.6 was reached the culture was induced with 0.5 mM IPTG for 3 hours at 37° C. The cells were harvested by centrifugation, lysed by a combination of the freeze-thaw method followed by disruption of cells with Bug-buster®, (Novagen). The lysate was separated by centrifugation into soluble (supernatant) and insoluble (pellet) fractions. Depending on the location of the protein different purification strategies were applied. A) If the His-tagged protein was in the soluble fraction, protein purification was done by binding the supernatant to Ni-Sepharose beads (Ni-Sepharose™ 6 Fast Flow, GE Healthcare). Due to the presence of the hexa Histidine (6×HIS) at the C-terminus of the expressed protein, it bound to the Ni-Sepharose while the other contaminating proteins were washed from the column by wash buffer. The protein was eluted by 500 mM Imidazole in 50 mM Tris, 0.5 mM NaCl buffer at pH 8.0. The eluate was concentrated, assayed by Bradford for protein concentration and checked by SDS-PAGE and Western blot. B) If the protein was present in the insoluble fraction the pellet was solubilized in suitable buffer containing 8 M Urea and applied onto the Ni-NTA column under denaturing conditions (in buffer containing 8 M Urea) using the same materials and procedure as mentioned above. Contaminating proteins were washed from the column by wash buffer without urea. Refolding of the His-tagged protein was performed while the protein was immobilized on the Ni-NTA matrix. After renaturation, proteins were eluted by the addition of 500 mM Imidazole. The eluate was dialyzed to remove traces of urea and concentrated if the volume was large, checked by SDS-PAGE and measured by the Bradford method.

### Animal Protection Studies

### Animals:

Male Sprague-Dawley rats (150 g)

### Active Immunization:

100 μg of recombinant protein combined with Freund's complete adjuvant (CFA) was injected subcutaneously, into the flank. Groups of six to ten rats were immunized with each antigen. Animals were boosted in the same area with the same amount of protein and adjuvant (Freund's incomplete adjuvant (IFA)), on days 14 and 28. Animals were monitored for any discomfort or lesions and were provided free access to food and water.

### Induction of Endocarditis:

One week following the final immunization, aortic valve endocarditis was produced in male Sprague-Dawley rats. The animals were anaesthetized using isoflurane for the placement of intravascular catheters. Hair was clipped at the surgical site and bupivicaine was administered (<1 ml/kg body weight) prior to performing surgery at this site. The right carotid artery was exposed, and a sterile polyethylene catheter was inserted via a small incision and advanced 4 cm into the left ventricle. Proper positioning of the catheter was ensured by sensing resistance and vigorous pulsation of the line. Blood was collected at the time of catheter placement and approximately 0.5 ml of immune sera was stored at −80° C. Following placement of the catheter, the catheter was heat-sealed and left in place during the course of the experiment and the skin was closed with sutures.

### Bacterial Challenge:

Twenty-four hours after catheterization, each rat was inoculated via tail vein with 10⁶-10⁷ colony forming units (CFU) (10× the infectious dose 50 (ID₅₀)) of E. faecalis strain OG1RF pre-mixed in 0.9% saline (0.2-0.4 ml volume). The animals were euthanized by CO₂ inhalation at 48 hours post-infection and the aortic valves examined. Vegetations were excised, weighed, and homogenized in 0.5 ml saline, and dilutions plated to BHI, BEA, and BHI+Rif100 agar for enumeration of CFU. Animals from which cultures of undiluted vegetations yielded no bacteria were considered to have had no induction of endocarditis. The number of infected and uninfected animals and of bacterial CFU recovered from non-immunized (control) versus immunized groups was compared to evaluate the protective effect of the antigens. Randomly picked colonies recovered from infected vegetations were also tested to re-confirm the identity of the infecting organism.

### Results

In the present invention, 20 E. faecalis antigens were tested in a rat catheter-induced endocarditis model. Groups of 6-10 rats were immunized with individual E. faecalis recombinant proteins combined with CFA (first immunization) or IFA (second and third immunizations). Of these, nine E. faecalis antigens showed a high level of protection (60-100%). In particular, 100% protection was observed in rats immunized with EF2703-1, 90% was observed in rats immunized with EF0032-2, 86% protection was observed in rats immunized with EF3177, and 71% protection was observed in rats immunized with either EF0792-1, EF1277, or EF3256-1.67% protection was observed for EF0270-2 and EF1060-1 and 62% protection was observed in rats immunized with EF3207. Partial protection (40-50% protection) was observed for seven proteins; EF0428, EF3051-1, EF1093-1, EF0779-2, EFC0034-1, EFA0042-1, and EF3183-1. Four proteins, EF1355, EF1026, EF1032 IB, and EF1692 appeared to show minimal protection (17-40%) (Table 3).

TABLE 2
DNA constructs made for heterologous expression. The protein name corresponds to the
nomenclature of the genes derived from the genome of E. faecalis strain V583.
The restriction sites (RE) used for cloning and the position (start/stop) of the
amplicon are indicated for each construct.

| | | | | DNA | Protein | SEQ ID NO |
|---|---|---|---|---|---|---|
| Construct | Protein | Vector | RE | (start/stop) | (start/stop) | (DNA, protein) |
| 1 | EF0032-2 | pET28b (+) | NcoI/XhoI | 1381/2613 | 461/871 | 1, 21 |
| 2 | EF0270-2 | pET28b (+) | NcoI/XhoI | 1348/1896 | 450/632 | 2, 22 |
| 3 | EF0428 | pET28b (+) | NcoI/XhoI | 1/1110 | 1/370 | 3, 23 |
| 4 | EF0779-2 | pET28b (+) | NcoI/XhoI | 970/1785 | 324/595 | 4, 24 |
| 5 | EF0792-1 | pET28b (+) | NcoI/XhoI | 115/657 | 39/219 | 5, 25 |
| 6 | EF1026 | pET28b (+) | BspHI/XhoI | 1/810 | 1/270 | 6, 26 |
| 7 | EF1032 IB | pET28b (+) | NcoI/XhoI | 1/2235 | 1/745 | 7, 27 |
| 8 | EF1060-1 | pET28b (+) | NcoI/XhoI | 82/1659 | 28/553 | 8, 28 |
| 9 | EF1093-1 | pET28b (+) | NcoI/XhoI | 97/1782 | 33/594 | 9, 29 |
| 10 | EF1277 | pET28b (+) | BspHI/XhoI | 1/480 | 1/160 | 10, 30 |
| 11 | EF1355 | pET28b (+) | NcoI/XhoI | 1/1617 | 1/539 | 11, 31 |
| 12 | EF1692 | pET28b (+) | NcoI/XhoI | 1/564 | 1/188 | 12, 32 |
| 13 | EF2703-1 | pET28b (+) | NcoI/XhoI | 88/1122 | 30/374^{a} | 13, 33 |
| 14 | EF3051-1 | pET28b (+) | AflIII/XhoI | 109/765 | 37/255 | 14, 34 |
| 15 | EF3177 | pET28b (+) | NcoI/XhoI | 1/789 | 1/263 | 15, 35 |
| 16 | EF3183-1 | pET28b (+) | AflIII/XhoI | 76/1074 | 26/358 | 16, 36 |
| 17 | EF3207 | pET28b (+) | AflIII/XhoI | 1/972 | 1/324^{b} | 17, 37 |
| 18 | EF3256-1 | pET28b (+) | NcoI/XhoI | 73/927 | 25/309^{c} | 18, 38 |
| 19 | EFA0042-1 | pET41b (+) | NcoI/XhoI | 121/801 | 41/267 | 19, 39 |
| 20 | EFC0034-1 | pET41b (+) | NcoI/XhoI | 10/1341 | 4/447^{d} | 20, 40 |

^{a}The residue Lysine at position 39 relative to the native protein was changed to an Isoleucine according to the sequence confirmation of the expression construct.
^{b}The residue Phenylalanine at position 40 relative to the native protein was changed to a Leucine according to the sequence confirmation of the expression construct.
^{c}The residue Lysine at position 237 relative to the native protein was changed to a Glutamic acid according to the sequence confirmation of the expression construct.
^{d}The residue Phenylalanine at position 236 relative to the native protein was changed to a Tyrosine according to the sequence confirmation of the expression construct. Mutations could be due to PCR amplification or be mutations in the particular V583 strain used for PCR amplification; SEQ ID NOs: 41-44 are fragments corresponding to the cases described under a-d, where such differences are not present.

TABLE 3
Protection conferred by E. faecalis recombinant proteins in a rat
model of catheter-induced endocarditis. Sprague-Dawley rats
(6-10 rats per group) were immunized subcutaneously with
100 μg of the respective recombinant protein cloned from
E. faecalis strain V583 adjuvanted with CFA/IFA. Rats were then
challenged intravenously with 10⁶-10⁷ CFU of E. faecalis
strain OG1RF. Rats with sterile cultures of undiluted vegetation
homogenates were considered to have had no induction of
endocarditis and therefore were protected. Fisher's exact test
was used to derive p values by comparing total number of
infected/non-infected by OG1RF versus infected/non-infected
by OG1RF + recombinant protein.

| | Protection | p-values of | |
|---|---|---|---|
| | (% animals in | protection | p-values of CFU |
| | which no | (infected vs. non- | treated vs. |
| Recombinant | infection was | infected) (Fisher's | untreated |
| proteins | detected) | exact test) | (unpaired t-test) |
| EF2703-1 | 6/6 (100%) | 0.0001 | <0.0001 |
| EF0032-2 | 9/10 (90%) | 0.0008 | 0.0002 |
| EF3177 | 6/7 (86%) | 0.0004 | 0.0001 |
| EF0792-1 | 5/7 (71%) | 0.0037 | 0.0044 |
| EF1277 | 5/7 (71%) | 0.0037 | 0.0045 |
| EF3256-1 | 5/7 (71%) | 0.0037 | 0.0112 |
| EF0270-2 | 4/6 (67%) | 0.0450 | 0.0424 |
| EF1060-1 | 4/6 (67%) | 0.0116 | 0.0113 |
| EF3207 | 5/8 (62%) | 0.0079 | 0.0049 |
| EFC0034-1 | 3/6 (50%) | 0.0631 | 0.0326 |
| EFA0042-1 | 3/6 (50%) | 0.0631 | 0.0315 |
| EF0428 | 3/6 (50%) | 0.2786 | 0.2305 |
| EF3051-1 | 3/6 (50%) | 0.0631 | 0.0813 |
| EF3183-1 | 3/6 (50%) | 0.0631 | 0.2920 |
| EF1093-1 | 3/7 (43%) | 0.1336 | 0.0367 |
| EF0779-2 | 3/7 (43%) | 0.0938 | 0.0072 |
| EF1355 | 2/6 (33%) | 0.2341 | 0.2394 |
| EF1026 | 2/7 (28%) | 0.6080 | 0.1468 |
| EF1692 | 1/6 (17%) | 1.0000 | 0.7784 |
| EF1032 IB | 1/6 (17%) | 1.0000 | 0.6286 |
| adjuvant-only | 1/6 (17%) | 1.0000 | 0.7528 |
| negative control | | | |

DNA sequences:
Construct 1: EF0032-2
(SEQ ID NO 1)
ATGGTCACTGGTATTTTAGATGATTGGAACTACGCATCACGCAGCTTGTATTCTGAGCCTTATCCTGACTTAAAA
AATCTAGTCGATCAAACAAAAAAAGAAAATGATACCTTTTACCGCTTGGAAAATTTAAATGGTGTTTCGGCCAAT
GACGGCATTAACTATGGCTACAGTGGCATTAGCATGTTTTCTTCCGTACGGAACCGACATTCTTCAACATACCTA
AACGCCTTAGGTTTCCGCTCACGAGGCACGAATTTAAATATTCGTTACCAAAATAATACCTTGTTAATGGATGCT
TTGATGGGCATTAAATACAATATTGCTGAAAACAATCCAATGAAATTCGGCTTTGAGCGACAAGCTGCTGCTGGT
AAATATCAGCTTTATCGTAACGAAAATGCTTTGCCGTTAGGATTTTTAGCAGACAAAGAGATTTACAATGTCCGG
CAACCTCTCAATGATAATTTAGGTAGTCAAACAAATTTACTAAATGCTTTAGCCAATACTAATGAACGGTATTTT
ACCTTTTATCAACCAACGATGACGCTTCAAAATAACGTGACAATTACGCAAAATACAGCTGGCGTAACTTTTACG
GAGAAACAACATAACGTAGCCAAAGAGATTTCTTATACAGTCAATGTGCCCGCCAATACGCAAGCTTATTTGAGT
TTATTCCCAACGGATTTTGCTCAATTGGAAAGTTCCACTGCGACGGTCACCGTGAACGGCTCAAGTCAGCAATCA
CAAATTGGCATTACTGGCCAATATTATAACTTAGGTTACTATCCCAAAGATACAACCGTTAACTTCAAAGTAAGT
TTTTATGGAACCAAAGCAGTAAGTTTTGTTCAGCCGCAAGTGGTTGGCTTAAATACCAATGCATTTGAAAAAGCC
ATTTCCGCCGTGCAAGAAAAAGGCGTTGATTTAACAACTGGCAAACGCTCTGCTTCAGGTACCTTTACCGCCGAC
AAAGATCAAGTACTCGTCACTACAATTCCTTATGACAAAGGTTGGCGTGTCAAAATTGATGGCAAAAAAGTTACC
CCAAAAGCCTTTAAAGATGCCTTTCTAAGCGTTCCTGTCAGCGCTGGAACCCATACTATTCAGTTCTCTTATTTA
CCAGAAGGATTGATTCCTGGCATCGTGTTATTCGTTCTATGTACTGGTGGTTTCGTTGCCTACGTAACCTTGATT
CCTGCTAGACGAAACCGTAAAAAAGAAGACAAA
Construct 2: EF0270-2
(SEQ ID NO 2)
GCCATTGCAGCGGTCGTTGGTTTTGGTCTAACATTCTTCTTCTGGAAAGACAATACAGTTGAGGAAGAAGAAGTG
ATTATTGATAAAACAACGATTAAAAAAGAAAATATTACAAGTCCAGTCAAAGGACGAGTGTTATCTTTAAAAAAT
GCAGAGGATCCTGCTTTTGCAAATGGAGCATTAGGAAATGGGGTCGTGATTGAACCAACGGAAGGTAAAGTGGTT
GCACCCTTTGATGGAACGATTGTCACACTATTCCCAACAAAACATGCATTAGGTTTGATTTCAGATAACGGCACA
GAATTATTGATTCATATTGGGATTGATACAGTTCAATTAGAAGGCGAAGGCTTTGAAGCTTTTGTAAAACAAGGT
GACCGCGTGAAAAAAGGTCAAACATTAGTAACCTTTGATTTAGAAGGAATTAAAAAAGCTGGGTTTAGTACACAA
ATTCCAATCGTTGTGACGAATACAGCGGATTATTTAGATATTTTGGAAGTCGGCAGTAATGAAGTTTCAACAAGT
GATGATTTGCTGACCGCTTTAATA
Construct 3: EF0428
(SEQ ID NO 3)
TTGCCGATGATTATTAAACAGGAACAATTCATACCAAAAGATACAGTAGAAACAACGATTGACTTATTAATTAGA
AATTTAACAACGATTAAAGATAACACAGGTGAATTTCTGTTAGATTTTGATGGTTTAAAAGTTGATGACAAAAGT
TGGACGATTTGGAACTGGCCGCAAGGCGTTGGGTTGTATGGTATATATAAGAATTATCGAAATACTAAAAGTGAA
AAAGCTTTACAAGTAGTTAACGACTGGTTTGAAGGTCGGATGCAAGAAGGCGCGCCGCCTAAGAATGTGAATACG
ATGGCTCCGCTTTTAACAATGGCTTATCTATATGAAGACACTAAAGACTCTAAATATATACCTTACTTAGAGCAA
TGGGCGGAATGGGTGATGGAAGAAATGCCCCGAACGAATGAAGGTGGCTTGCAACATGCGACATATGGACCAGAA
AATAAAAATCAATTATGGGATGATACGTTAATGATGACTGTCTTACCTTTGGCAAAAATTGGTAAGTTGTTGAAT
CGACTCGATTATTTGGAAGAAGCAAAGCATCAATTTTTAATCCATATTAAATATTTACAAGATAAAAAAAGTGGT
CTATGGTATCACGGTTGGACTTTTGAGGGAAATCATAACTATGCGGAAGCACTGTGGGCAAGAGGAAACTGCTGG
ATTACGATTGCTATTCCAGAAATTATTGAAATCTTAGAGTTGCCAAAAGGAGATAGTTTACGGGAATTTTTATTG
AGTACCCTCAATGCGCAAGTGGCAGCTTTAGCGAAATACCAAGATGAATCTGGTTTATGGCATACATTAATTAAT
GATTCAAATTCGTATTTAGAATCTTCTGCTACAGCGGGATTCGCTTATGGGATTTTAAAAGCGGTTCATAAAAAA
TATATTTCTTCTGAATATGAAGAAGTGGCAAACAAAGCAATTGCTGGCTTACTAAATGAGATTGATGAAACGGGA
GAAGTACAACATGTGTCAGTTGGTACAGGAATGGGTGATAATTTAGATTTTTATCGCACAATTGGAATGACAGCG
ATGCCTTATGGTCAATCATTAACAATCCTATGTTTGACTGAATTGCTTGTTTCTTATTGC
Construct 4: EF0779-2
(SEQ ID NO 4)
GGCTATATAATTTTCAACGCTGTTTACTTAACAGGGCTATTAGAATCAAAACCTTTGATTATTTCGCATCGAGGG
GTCACCAATAGTAATGGCGTACAAAATACCATTCCTGCTATGGAAAGAACCATTAAATTTAAACCAGATTATATT
GAAATTGATGTTCAAGAAACCAAAGACCATCAATTTGTTGTGATGCATGATGCAAACCTTCAAGAACTCGCCGGC
GTTGATGGCACGCCGCAAGAATTTACGTTGGCTGAATTAACAAAAATGACCGTTAAAGAAAATGGCCAAGAAGCA
CCAATTGCCAGTTTTGATGATTATTTAGCGAAAGCAAACCAAGCCAAACAAAAATTACTAGTAGAAATTAAAACG
TCTAAACAAGATAGTCAAGGTGCGCTTTCTAACTTTATTGAAAAATATGAACGTCCTCTGATAAAGAATAACCAT
CAGGTTCAATCTCTCGACTACAATGTGATCAAAGCGTTTAAAAAAGCCAAATCAAAAGTAAAAGTTAGTTTTATT
TTGCCCTATAATTTCACTTTTCCAGAAACACAAGCTGATTTGTATACAATGGAAGCTACTACGTTGAATGATACC
TTCATTTTAAAAGCTGACCAACAGAAAAAAGCAGTTTATGCTTGGACAGTTAATGACTCAGAGGTATTGAGTAAA
ATGCTCTTTATGGACGTTGCTGGAGTGATTACCGATGATTTGGAGTTAGTCAATGAAGAAGTCAATGACTTTGAA
AAAAATCCGTCGTATGCGGATCGTATTTTGCATTATATTTTTATGCTACCAAGTGTGGCTTCTCAA
Construct 5: EF0792-1
(SEQ ID NO 5)
TTAGCTCAAGGAAATCGTTTAGCCGTAGATCAATGGCAAGCGAATCAAGTGGTTTTATCAAAAGAGGCCAATAGT
AATTTAAATGTATCAGTGTTAGATGAAAACGTGAAAGAAACGATTTCAGGAGGCAAAATTGCACCGATTGGTCAA
CAATCTTTAGCCATCCGCCCAGCAGATGATAAAAAGGCTGAATTAACGAATGTTAGCTTATTTGGGATTGAAAAA
GAAAGTTTTTTAATGCCAAAAGTGATTGAAGGAAACGCATTTACTGATAAAAATCAAGTGATTGCTTCAGAAACA
TTGAAAAATCAAGGATTTAAAATTGGCGACAAATTAACTGCAGGGAAATATGATGAGCAATTAGAAATTGTCGGC
TTTATTTCTAAAAGTAGCTATAACATTGTGCCAGTTATTTACACTTCTCTGGATACTTGGCGGTCAATTAAATAT
GGTGACAATCCAGCAATGGCCAAAATGGTTAATGGTTTCATTGTTCGCAGCAAGGACAACACGGAAGTTAAAACG
ACTAATAAAGACAGCCAA
Construct 6: EF1026
(SEQ ID NO 6)
ATGAAAAAAGAGATCATTGTTTATACTATTTCCGATTCACTTGGAGAAACATCACAAAAATTGTTAGCTGCAGCA
AGCGCACAATATCCAGATATTTCTTTTCTTAATCGATACAATTTTTCTTTTGTCACAACAGAAGAAGAATTATTA
GAGATTTTAAAAGATGCCTTAAAAGATAAAGCCTTAGTTGTCAGTACATTAGTCAGTAAACAACTAATCACAGCG
GCGAAAGAATTTAGTGAACGAACAGGGTTGTTATATTTAGATTTAATGGCGCCATTTTTTGAATTAATTCAAGCG
AAAGCCGGAGTAGATCCTATTGAAGAGCCTGGACGACGCCACCAACTAGATCGTGCCTATTTTGATAAAATCTCA
GCGATTGAATTTGCTGTAAAATATGATGATGGCAAAAATCCTCAAGGGTTTCTTGATTCTGATATATTGTTGTTA
GGCGTTTCGCGGACCTCAAAGACGCCAGTCAGTATGTATTTAGCGAATCAAGGCTACCGCGTTTCTAACTTACCA
TTAATTCCAGAAGTTCCATTGCCGCCAATTTTGGAAGAAATGGATCCACAAAAAATGATTGGTTTAGTTTGTTCG
CCAGAAACATTAGGACAGATTCGTAGCAGTCGGTTGGCTTCCTTAGGTTTAGGTAATGAGACCAGTTATACCAAT
GTTGAACGGATTGAACAAGAATTAGCTTATGCCGAAGAGATTTTTGCGAAGTATGGCATCCCAGTGATTGATGTA
ACCGCAAAATCTGTCGAAGAAACAGCCTTTTTAATTAAAGAAAAACTAGATGAAAGAAAT
Construct 7: EF1032 IB
(SEQ ID NO 7)
ATGGAATGGATTGAAATTAAGCACGCAACACAAAATAATTTGAAGAATATCTCTGTCAATATCCCTAAAAAGCAA
CTAACTGTTGTTACTGGACTTTCGGGTTCAGGAAAGTCCTCCTTAGTATTTGACACATTAGCCGCTGAATCACGT
CGTGAACTAAATGATACATTTAGTTCGTTTGTTCAAAATTACTTACCCAAATATGGTCGTCCAGAAGTTGAAAAA
ATCGAGAATCTTCCTGTTGCAATTGTCATTGACCAGAAAAAAGTAGCAGGTAATTCTCGTTCGACAGTGGGAACG
TACACAGATATTTATACGTTTTTACGTTTGCTGTTTTCACGAGCAGGTTCTCCATTTGTTGGTTATTCAGATACT
TTTTCGTTTAATCATCCGGATGGAAAATGTCCAACATGCGATGGCTTAGGAAAGATTACCGAAATCAATCTTCAT
CAGCTAGTTGATTATGACAAATCATTAAACAAAGGGCCTATCGATTTCCCCACATTTACAGTTGGCAACTGGCGG
TGGAAGCGTTATGCCCATAGTGGTCTATTTGATTTGGATAAAAAAATCAAGGACTATTCTCCTGAAGAGTTAGCA
TTATTTTTATATGCTCCACAACAAAAACTAGCTAATCCACCCAAAGAGTGGCCTCATACAGCTTTGTATGAAGGA
ATCGTCCCGCGTATGCAACGTAGCATATTGCATACAGACGAAGGCAAACGTCATCAAAAATACCTTAATCACTTT
GTTACCGTAAAAAGATGTCCTGATTGTTTAGGAAGTAGAGTCAATGAACGTGTTCGTAGCTGCAAAATTAATCAG
AAAAGTATTGCTGATGCTGTTGACATGCCACTCACTGAATTACATTCTTTTATTCGTTCAATGGACCTATCCTTA
ATAAAAACTATTCAAGAAGAGCTACTTGTACGTCTAGAAGCATTAATTAATATCGGTCTCTCCTACCTCACATTA
GGACGAGCAACTGAAACACTTTCTGGTGGTGAAGCACAGCGGATTAAAATTGCTAAGTATGTAAACAGCGCCTTA
AATGATATTATGTATATTTTAGATGAACCAAGTGCTGGCTTACATCCAAAGGACATCGAACGGATCAGTCGTGCA
TTGCTCAATTTAAAAAATAAAGGAAACACCGTGGTTCTCGTGGAACATAATCCACAATTAATTAGAGAAGCTGAT
TTTATCATCGATATCGGACCTTTCGCAGGCGAAAATGGTGGCCATGTCCAGTTTTCAGGAACGTATGACGCATTT
TTAGTCTCCAAAACCTTGACTAGTCAAGCGCTTCAAGAGCCGCTCCCTTTAAACGACCAACCAAGAAAAGCAAGG
AAGTCTTTATCAATCGAACATGCGACACTGCATAATTTAAACAATCTATCTGTCGAAGTTCCGTTAGGAGTTTTG
ACTGTTATTTGTGGGGTCGCTGGTTCAGGTAAATCATCATTAGCGGAAGAAATTTATCAAAAAGCCCAGGCTGAT
AACCAAGAAATTATTCATCTTTCACAAAAAAGCATTACCGCAAATTTACGATCCACACCTATGACCTATCTTAAT
ATTTTTGATAAGGTCCGCAAACTGTTTGCGGAAGAAAATCATGTTAGTCCAGCTTTATTTAGCTATAATTCCAAA
GGCGCCTGTCCTACTTGCAAGGGGAAAGGCATAATTGTCTCCGATATGTCTTTTATGGAGGATGTTACTAGTATT
TGCGAAACCTGTCACGGAACGCGTTACAAAGAAGAGGTGCTTCATTATCTGTATAACGGAAAAAATATCGTTGAA
GTACTAGCTTTAAGCGTTAAAGATGGCTATGATTTTTTCAAAGACCAGCCTTTTGCTCTTTCATTAAAAAATTTA
CTGGAAGTTGGCTTAAGCTATCTTAAACTCAATCAATCGCTCTCAACATTATCTGGTGGCGAATTGCAAAGGGTA
AAATTAGCAGACACACTTCACCAAAAAAAAGCTATCTATTTAATGGATGAACCTACAGATGGTTTACACTTAATT
GATATCCAACAAAGTCTTCAACTTTTCAATCGAATGGTAGAAGAAGGAAACAGCTTGATTTTATTAGAACATCAC
ATTGATGTGATTAAAAGTGCCGACTGGTTGATTGAACTAGGTCCCGAAGGTGGAAAAAATGGCGGACAGCTTCTT
TTTACAGGAACCCCAGCAAATATGCTAAATTCCACTCATTCTATTACTAAAGGCTATCTG
Construct 8: EF1060-1
(SEQ ID NO 8)
GATAGTAACAAAGCAGCGGAACAAAAAATTGCAATTAGTTCTGAAGCGGCTATTTCGACAATGGAACCACACACA
GCGGGGGATACGACCTCGACTTTAGTCATGAATCAAGTTTATGAAGGACTCTATGTTTTAGGTAAAGAAGATGAA
TTAGAGTTGGGGGTCGCTGCCGAAGAACCAGCGATTTCTGAAGATGAAACCGTTTATACATTTAAGATTAGAGAA
GATGCCAAATGGTCGAATGATGATCCAGTAACAGCAAACGACTTTGTTTATGCATGGCAACAAGTTGCTTCCCCT
AAATCAGGATCGATTCATCAAGCTTTATTTTTTGATGTCATTAAAAATGCTAAGGAAATTGCTTTAGAAGGCGCA
GATGTGAATACTCTTGGGGTTAAGGCGCTAGATGATAAAACGTTAGAAATAACTTTAGAACGGCCCACCCCTTAT
TTGAAATCATTACTTTCGTTTCCTGTTTTGTTTCCACAAAATGAAAAATATATCAAAGAACAAGGGGATAAATAT
GCTACTGATGCAGAACATTTGATTTATAATGGTCCTTTTAAATTGAAAGAATGGGATAATGCCTCTTCTGATGAC
TGGACCTACGAAAAAAATGATACGTATTGGGATGCTGAAAAAGTTAAATTAACAGAAGCGAAAGTTTCAGTAATT
AAGAGCCCAACGACAGCGGTGAATTTGTTTGACTCGAATGAATTGGATGTAGTGAATAAGCTAAGTGGTGAATTT
ATTCCTGGTTATGTTGATAATCCAGCCTTTCTTTCAATTCCTCAATTCGTCACATACTTTTTAAAAATGAACAGC
GTTCGTGATGGAAAAGAAAATCCGGCTTTAGCGAACAACAATATTCGTAAAGCGTTGGCACAAGCTTTTGATAAA
GAAAGTTTTGTAAAAGAAGTCTTGCAAGATCAATCAACGGCTACAGATCAAGTAATTCCGCCGGGACAAACGATT
GCGCCAGATGGAACAGATTTCACAAAACTAGCTGCTAAGAAAAATAACTACTTAACCTACGATACAGCGAAAGCA
AAAGAATTCTGGGAAAAAGGGAAAAAAGAAATTGGGCTGGATAAAATCAAATTAGAATTTTTAACAGATGATACA
GACAGCGCCAAAAAAGCTGCTGAGTTTTTCCAATTTCAATTGGAAGAAAATCTAGATGGATTAGAAGTGAATGTT
ACTCAAGTTCCTTTTACTATTCGTGTTGATCGTGATCAAACGAGAGACTATGATTTAGAATTATCTGGTTGGGGA
ACCGATTATCGTGATCCATTAACAGTTATGCGCATCTTTACTTCGGATAGTACCTTGGGCGGCGTAACGTTCAAG
AGTGATACGTATGATCAATTAATTCAAGAAACTAGAACAACACATGCGGCTGATCAAGAGGCTCGTTTAAATGAC
TTTGCTCAAGCACAAGATATTTTGGTGAATCAGGAAACGGTTTTAGCACCAATCTACAATCGAAGCATTTCTGTA
TTAGCTAATCAAAAAATCAAGGATCTGTATTGGCATTCATTTGGACCCACGTACAGTTTAAAATGGGCTTATGTT
AAC
Construct 9: EF1093-1
(SEQ ID NO 9)
GAAGAAAATGGGGAGAGCGCACAGCTCGTGATTCACAAAAAGAAAATGACGGATTTACCAGATCCGCTTATTCAA
AATAGCGGGAAAGAAATGAGCGAGTTTGATAAATATCAAGGACTGGCAGATGTGACGTTTAGTATTTATAACGTG
ACGAACGAATTTTACGAGCAACGAGCGGCAGGCGCAAGCGTTGATGCAGCTAAACAAGCTGTCCAAAGTTTAACT
CCTGGGAAACCTGTTGCTCAAGGAACCACCGATGCAAATGGGAATGTCACTGTTCAGTTACCTAAAAAACAAAAT
GGTAAAGATGCAGTGTATACCATTAAAGAAGAACCAAAAGAGGGTGTAGTTGCTGCTACGAATATGGTGGTGGCG
TTCCCAGTTTACGAAATGATCAAGCAAACAGATGGTTCCTATAAATATGGAACAGAAGAATTAGCGGTTGTTCAT
ATTTATCCTAAAAATGTGGTAGCCAATGATGGTAGTTTGAAAAAAGTAGGAACTGCTGAAAATGAAGGATTAAAT
GGCGCAGAATTTGTTATTTCTAAAAGCGAAGGCTCACCAGGCACAGTAAAATATATCCAAGGAGTCAAAGATGGA
TTATATACATGGACAACGGATAAAGAACAAGCAAAACGCTTTATTACTGGGAAAAGTTATGAAATTGGCGAAAAT
GATTTCACAGAAGCAGAGAATGGAACGGGAGAATTAACAGTTAAAAATCTTGAGGTTGGTTCGTATATTTTAGAA
GAAGTAAAAGCTCCAAATAATGCAGAATTAATTGAAAATCAAACAAAAACACCATTTACAATTGAAGCAAACAAT
CAAACACCTGTTGAAAAAACAGTCAAAAATGATACCTCTAAAGTTGATAAAACAACACCAAGCTTAGATGGTAAA
GATGTGGCAATTGGCGAAAAAATTAAATATCAAATTTCTGTAAATATTCCATTGGGGATTGCAGACAAAGAAGGC
GACGCTAATAAATACGTCAAATTCAATTTAGTTGATAAACATGATGCAGCCTTAACTTTTGATAACGTGACTTCT
GGAGAGTATGCTTATGCGTTATATGATGGGGATACAGTGATTGCTCCTGAAAATTATCAAGTGACTGAACAAGCA
AATGGCTTCACTGTCGCCGTTAATCCAGCGTATATTCCTACGCTAACACCAGGCGGCACACTAAAATTCGTTTAC
TTTATGCATTTAAATGAAAAAGCAGATCCTACGAAAGGCTTTAAAAATGAGGCGAATGTTGATAACGGTCATACC
GACGACCAAACACCACCAACTGTTGAAGTTGTGACAGGTGGGAAACGTTTCATTAAAGTCGATGGCGATGTGACA
GCGACACAAGCCTTGGCGGGAGCTTCCTTTGTCGTCCGTGATCAAAACAGCGACACAGCAAATTATTTGAAAATC
GATGAAACAACGAAAGCAGCAACTTGGGTGAAAACAAAAGCTGAAGCAACTACTTTTACAACAACGGCTGATGGA
TTAGTTGATATCACAGGGCTTAAATACGGTACCTATTATTTAGAAGAAACTGTAGCTCCTGATGATTATGTCTTG
TTAACAAATCGGATTGAATTTGTGGTCAATGAACAATCATATGGCACAACAGAAAACCTAGTTTCACCAGAAAAA
GTACCAAACAAACACAAAGGTACCTTACCTTCAACA
Construct 10: EF1277
(SEQ ID NO 10)
ATGAACATTTTTGCTGTCCGTTTAAAAGAGGCTTTAACAGCCAAGAACATCAAACCTAGTGATTTAGCGAAAAAA
ACTGGCATCGGTAAATCTTCGATCAGCGATTGGCTAGCTGGTCGTTACGAAGCAAAACAAGACAAAGTTTATCGC
ATTGCAGATGCATTAGACATTAATGAGGCCTGGTTGATGGGACAAGAAGTTCCCATGGAAAAAAATGCCTCAACT
ATCGACCGCATTTATAAAAAATTAGAACCCCAACGACAAGCCATCGTTTATCAATTTGCCGAACAACAATTACAC
GAACAACAAACGCAAGCAGAAATTCTCTCATTCCCTCGCCGTGACGAAATGACACTGGCTGCCCACGCTGGGGAT
CCAGAAAAGATATTTTCAAAAGAAGAGATCGAGAAAATTCACGATTACTTGGATGAAATTGATGCCAAATATCAA
CAATCGATTTCTTCTGACAAAAAAGAGGAT
Construct 11: EF1355
(SEQ ID NO 11)
ATGGCTTATCAGTTTAAATTACCGGATATCGGTGAAGGGATTGCCGAAGGCGAAATCGTTAAATGGTTTGTAAAA
CCTGGCGATACAATCAACGAAGACGATACGTTATTAGAAGTACAAAATGACAAATCAGTGGAAGAAATTCCATCA
CCAGTAACAGGTACTGTAAAAAATATCGTTGTACCAGAAGGAACAGTTGCAAACGTTGGTGACGTGTTAATCGAA
ATCGACGCACCTGGTCACGAAGATAACGATGCAGCACCAGCAGCTCCTGCACAAGAACAAACACCAGCACAACCT
GCTGCTGTACCAACAACCGAAGCAGCTGGCGGATTTTTCCAATTCAAATTACCAGACATCGGTGAAGGAATTGCC
GAAGGCGAAATCGTTAAATGGTTCGTTAAAGCGGGCGACACAATTAATGAAGATGATTCATTATTAGAAGTACAA
AATGACAAATCAGTAGAAGAAATTCCATCACCAGTAACAGGTACTGTAAAAAATATCGTTGTACCAGAAGGAACA
GTTGCCAATGTGGGTGACGTGTTAGTTGAAATTGACGCACCTGGTCATAATTCAGCAGCACCGTCAGTCGCAGCA
CCAGCTACTGACGCTCCTAAAGCGGAAGCATCAGCTCCAGCCGCTTCAACAGGCGTAGTTGCAGCCGCTGATCCA
AACAAACGCGTTTTAGCAATGCCATCTGTTCGTCAGTATGCGCGTGAAAAAGACGTTGATATTACACAAGTAACT
GCAACTGGTAAAGGTGGCCGTGTCATTAAAGCGGATATTGATGCCTTTGTCTCTGGTGGTTCTCAAGCAGCCCCA
GCTACTGAAGCTGCCGCAACAGAAGCAGCACCTAAAGCGGAAGCAGCTGCACCTAAAGCAGCGCCAAAAGCCTTT
ACTTCTGATTTAGGCGAAATGGAAACACGTGAAAAAATGACACCAACACGTAAAGCAATTGCTAAAGCAATGGTT
AACAGCAAACACACTGCTCCTCACGTAACATTACATGATGAAGTAGAAGTTTCTAAATTATGGGATCACCGTAAG
AAATTTAAAGATGTTGCTGCTGCAAATGGTACAAAATTAACATTCTTACCATACGTTGTAAAAGCATTGACTTCA
ACTGTTCAAAAATTCCCAATCTTGAATGCATCAATCGATGACGCAGCACAAGAAATTGTTTACAAAAATTACTTT
AACATTGGTATCGCTACTGATACAGATCATGGCTTATATGTACCAAATGTTAAAAATGCTAATACGAAGAGCATG
TTTGCTATCGCTGATGAAATCAACGAAAAAGCAGCATTGGCTATCGAAGGTAAATTAACTGCACAAGATATGCGT
GATGGTACAATCACAATTAGTAACATTGGTTCAGTCGGTGGCGGCTGGTTTACACCAGTAATCAACTACCCTGAA
GTTGCTATTTTAGGCGTTGGTACAATTGCACAAGAACCAGTTGTTAATGCAGACGGCGAAATCGTTGTGGGACGC
ATGATGAAATTATCATTAAGCTTTGACCACCGTATCGTTGACGGCGCAACTGCTCAAAAAGCAATGAACAACATT
AAACGCTTATTAGCTGATCCAGAATTACTATTAATGGAAGGA
Construct 12: EF1692
(SEQ ID NO 12)
ATGGCAAAACGAATTATCATTATGAACTTTGATATTGAAAGTAAATCGTACCAAGCATTTTCCGAAATAAAAAAG
ATGCAGGCAGAAAGACAGCTTAAAGGTGAGCAAATGGCGGTTGTTACGCATGTTAATGATGGCCAGCATCAATTT
AAAATCAATGATTTTATCGATTTTACAGGCAACAATCATACATCAAAAGATAGCATGATTGGGATGCTAGTAGGC
ATATTAGGTGGTCCTCTGGGCATTCTTTTTGGCTGGTTTGCTGGAAGTATGTATGGTGCAAGCAAAGACGCCAAA
GAAATTCAAGAAGCACAAACGGTTTTTGAACATGTGATTCAAAAGATTGATGAAGGACAAACGGGATTGTTATTA
ATTGCAGAAGAAGAAGACAACCGTCCGCTCAACCAATTGGTTATGTTTGACTTAGGTGGCGAAATCACGCGGCTT
GATTTAGAGGAAGTCCAACAAGAAATTAACGATGCGAACGAAGTTGCAAATGAAGCGAAACAATCGTGGCAAGCA
AAAAAAGAACAACACAAAGAAGCAACATCGAAAGAAGAA
Construct 13: EF2703-1
(SEQ ID NO 13)
GGTAGCTATTTAAAGAAAACAATTGATATAGGCTATGTTCCCATAAAAAATGATTATAATGAAGCGCAAAATAAA
GATAGTCAATCGTTTTTGATTATGGGGCTAGACAATACAATTGAACGGAAATTAGGCACAACTAGGACTGATGCT
ATGATGGTGATTACCGTGAATAACAAGACGAAGAAAATAACCTATTTAAGTTTGCCACGGGATAGTTTTGTTCAA
ATTGATGCGAAAAATTACCAAGGGATGCAGCGAATTGAAGCCGCCTATACCTACGATGGACCAACAGCTTCTGTT
AACACAGTTGAGAAATTATTGAATATTCCAATCAATCATTACGTTGTGTTTAACTTTTTATCTTTTATTAAGTTA
ATTGATGCGGTTGGCGGCATAGATGTCAATGTCAAGCAGGCGTTTGATGGTGTCACCAAAGACGGGCCAGGATCC
ATTCATTTTGATGCAGGGAAACAGCATTTAGATGGTACGAAAGCTTTATCTTATGCCCGTGAAAGACATAGCGAT
AACGATATTATGCGTGGATTCCGACAACAAGAAATTATTCAAGCAGTTGAAGACAAGTTGAAATCTGGTCAATCA
ATCATGAAAATAATGGACATTATTGATTCGTTAAATGGAAACATTCAAACTGATGTGGATTCCAATGAATTGACT
CATTTAGTCAAAGAAGGTTTGACTTGGACCAATTATGATAAACAACAGCTTTCTTTTGACTGGCGCACTTTTAGT
AATGAAGGGCGCAGTATGGTTGAACTATACCCAGATAGTATTGAAAATGTCCGTCATCAATTACGTGTGTCTTTA
AATTTAGAAAAGCCAGATGAACGAGATCAAGACGGCTATGTCTTCCATACGAACGGTGAATTTTTATATCAAAGT
GATTATACCGTTCAAGATGAAGCAGCTGAGGAAAACGAAATGACTTCCATCAACGGCAATACGTATATTGGTGTT
CCTGGTAATACACAGACCGGCCCGTTGCCATCAGTTAAAACGGAAAATGGCTTTATAAAA
Construct 14: EF3051-1
(SEQ ID NO 14)
TTTTCAAGTTGGTTTGATGATTCATATGTCAAAGCTAGTGCTGAATCAAGTAAAACAAAAGAACCAGCACCAGTT
AAAATTGAGAAAAAAGTCAAACCTTTAAGCTATGGACAACAGGTCAATCAAGAAATTGAAAAGAAGCAATATGAT
GGACATCTGGATTTGCCGTTAGAATTGCAGACAGATGCTAAATGGAAAGACACCGCATACGGATTTGGCAATGTG
GATAAGCCGAATACAATCGAAATTAATGGCTGTGCGATTGTATCGCTTGCAATGGTTGGTTCATACATGGATCAC
CAAGAAGTTACCCCTCTGGATGTGTTAGCTTGGGCAAAAAATGACTTCTTTATGGAAGGGCAAGGGACGGCGTGG
TCTATTTTTAGTGCATATGCTGAAATGAAAGGCTATAACTGTCAAGAAATTGGGGATATTGAAACAGTGGCAGCT
TTCTTGAAGGAAGGTCATCCAGTCATTATTTCTGTAAAACCGGGCTATTTCACTACAACTGGTCACATTATGGTG
ATGAGTGGTGTGGATGAAAAAGGCGATTTCTGGATTAATGATCCAAACGATTCAGAAGAAAAGGGCCATTCAAAA
CGGACATTTACAGCCGAAGAAGTGATGAATGAAGCGTTAAACTTCTGGGCATTTTAT
Construct 15: EF3177
(SEQ ID NO 15)
ATGGACTTACATTTAACCGTTGATCTAAAAAAAACAAAGACACTCATTGACTTAGCCGATCAGGCCCACTTAATC
ATTGAACAAACAACTAAATTACCGAAAAAAGAGGCAGCGATTCTTTTCTTGCTAATAGATAACCAACTAGTTGCT
TTAGGACTTGCTGAAGAAAAAGCTACATATAAAGAAGTTTCTTTTGATCACTCGATTTTATTGAAACCGACTTGG
GACACAGAACTTGCCTATTTAGCGCAAGCTTTCCTAGATGATGCGAAAGAAAGTGGTTTAACATTGGAAGCAACC
CCAACGACAGTCAAAATTCCGAAAAGTGCGGTAGCGACAGTGACTAACTATAAAGAAACAGTCACCTTTATCTTG
GAACGTTTTGGCTACTCTCTGTTTAAAAAACCGGCGCCGAAGAAAGCCAGACCAGCCAAAGCACGCCACAAATGG
ACAAAAGAAGTTAGCCAAATTCCTTTTTATATTGATACTCGACAAAGCAAAGCAACTGTTTTTTGGCAAAAGCGC
AATGAAATGCTTATTAAAGCTGGCGCGACAATGATGCCCGAAGCCCCTTTAAATAAAGACGGGTCTGTCGGTTTC
TCCGCTAGATTTGGTGAAAAACTTCGTGATGAACGCAAGGGCCAATTTAAAGACTTTGTCACAACGGAAGATATC
GTCTTAAAAAGCGTTAATGAAGTCGGCTTGTTTCTCTATTTTGCTGGAACAAACAGCTGGTTAGAGCTAGTTGAT
GAGAACGGCAAAACCTTAAATGAATGGACTGTGGTGGAA
Construct 16: EF3183-1
(SEQ ID NO 16)
TCTGAATTTAATTTTGCGGTCACACCAACAATTCCCGAAAATCAAGTGGATAAATCAAAAACCTACTTTGACTTA
AAAATGGCGCCTGGTGCCAAACAAACCGTAGAAATTCAGTTACGCAATGATACAGATGAAGACATTACCATTGAA
AATACGGTGAACTCAGCGACAACAAATTTAAATGGCGTAGTAGAATATGGCCAAAACGGGATCAAACCTGACAAA
ACCTTACGTTTTAACTTAAAAGATTATGTGGAAGCACCGAAAGAAATCATCTTGCCGAAGCATTCCCAAAAGACC
TTACCTTTAACCATTACGATGCCTAAAGATTCTTTTGATGGCGTGATGGCTGGCGGTATAACACTCAAAGAGAAA
AAGAAAGAAACAACGACTTCTGCGGATCAATCAAAAGGGTTAGCTATTAATAATGAATACTCCTATGTTGTGGCT
ATTATTCTTCAGCAAAATGAGACAAAGGTTCAACCAGATTTAAAATTACTGGGGGTTAAACCAGGCCAAGTCAAC
GCGCGAAACGTCATCAATGTTTCTTTACAAAACCCACAAGCGGCCTATTTAAACCAATTACATTTAATCAACACT
GTTTCAAAAGGAGGCGAAACGCTTTACCAATCCGATACTGAGGATATGCAAGTGGCGCCAAACTCTAACTTTAGT
TACCCAATTTCTTTAAAAGGGGAACGATTAACGCCAGGAAAATATGTCTTGAAATCAACGGCCTATGGTGTAAAA
GATGAAAAGGGCACCTATCAAGTCAAAGGCGCCAATGGTGAAGAACGGTACCTGTACAAATGGGAATTTACAAAA
GAATTTACTATTTCTGGGGACGTCGCTAAAGAATTAAATGAAAAAGACGTAACCATTAAAGGAACCAATTGGTGG
TTGTATCTACTGATTGCATTAATCATTCTAGCGCTGCTCTTATTGATTTTCTTCTTGTATCGTAAAAAGAAAAAA
GAGGAAGAACAACAATCTGAGCAA
Construct 17: EF3207
(SEQ ID NO 17)
ATGTCCAAGAATTTTTGGGCAACATTGCCAAAGCCCTTTTTTGTTTTAGCACCGATGGAAGATGTCACTGATGTG
GTCTTTCGTCATGTCGTGAAAGAAGCTGGGGCACCAGATGTGCTTTTTACAGAATTTACTAATTCGGATAGTTAT
TGTCATCCTGAAGGAAAAGATAGTGTACGCGGGCGCCTCGTTTTTACAGAAGATGAACAGCCGATGGTGGCACAT
ATTTGGGGGGATAAACCGGAATTTTTCCGTGAAATGAGTATCGGCGTAGCGGAGATGGGCTTTCAAGGGTTAGAC
ATAAATATGGGCTGTCCTGTGCCTAACGTGGCTGAGCGTGGAAAAGGCAGCGGGCTAATTTTGCGCCCAGAAGTC
GCGGCTGAATTGATTGACGCTGCCAAAGCAGGTGGCTTACCTGTTAGTGTCAAAACACGCATTGGGTTTACTGAA
ATGGCGGAAATGGAGGCGTGGATCACGCATTTATTAGAGCAAGACATTGCGAATCTATCCATTCATTTGCGAACA
CGAAAAGAGATGAGCAAAGTGGATGCCCATTGGGAGGTCATTCCGCAAATTATGGCTATTCGGGACCGTGTCGCA
CCGCAAACGACAATTACGATTAATGGGGATATTCCCGATCGTCAAAAGGGCCTAGAATTAGCAGAACAATATGGT
GTAGATGGCATCATGATTGGTCGGGGGATTTTTAAAAATCCTTATGCCTTTGAAAAAGAACCCAAAACACATACG
CCACAAGAATTGCTGGGCTTGCTACGTTTACAATTGGATTTGCAAGACAAATATGCGGAATTGGTGCCTCGCTCC
ATCGTTGGGCTGCATCGCTTCTTTAAAATTTATGTCAAAGGCTTTCCAGGTGCCAGTGATTTAAGAGCACAATTA
ATGAATACAAAATCAACGGATGAAGTGCGCCAGTTGTTAGCGACGTTTGAAACAGAACATGGTGTGCTTGAC
Construct 18: EF3256-1
(SEQ ID NO 18)
GCAGACAAGAAAGACAACACAACGAACTCTTCTAGCGCAGCATCTTCAGAAACGAAAAAATCAACGGAATCATCA
GCACCAGCGAAAAAAGTTGCCGGTGGCGATTTAAAAGATGGTACGTATAAATTAGAAGAAAAAAATGAAAAAAAT
GGTTACCGTGCAGTCTTTGAAATGACTGTAAAAGACGGCAAAATCACTGAATCTAAATATGACAACATCAATGCT
GACGGCAAGTCTAAAACAGAAGACACTAAGTATGAAGAAAGCATGAAAGCAAAATCTGGTGTTGGACCAAAAGAA
TACATCAAACAATTAAACGATTCTTTTGTTAAAGCACAAAGCGCAAGCGGTGTGGAAGTAGTAACTGGTGCGACT
CATTCATCTGAATCATTCCAAAACTACGCACAACAATTAATCCAAGCAGCACAAGCTGGTAACACAGACACAATC
GAAATCGACAATGGGGCAACATTGAAAGATGGTACGTACTCATTGAAAGAAAAAAATGACTCAAACGGCTACCAC
ACAACATTCTCAATGACTGTGAAAGATGGTAAAGTGACTGAATCTAACTACGATAACGTGAACGCTGACGGCAAA
TCTAAAAAAGATGACACTGAATACGAAAGCAAAATGGAAGACGTTGCTGGCGTTGGACCAAAAGAATATATCGAA
ACATTAAACAAAGAATTTGTTAAAGCAATGGGCGAAGAAGACGGATCACCTGCAGGTGTTGAAGTAGTAACTGGT
GCAACACACAGCACACATTCATTCATCAACTACGCACAACAATTAGTGAACGCTGCTGAAAAAGGCGACACAACT
GAAATCGTTGTTGACAACATCGTAACAAAA
Construct 19: EFA0042-1
(SEQ ID NO 19)
TTTGCACAAGAAATTATCCCTGATGATACTACGACACCGCCCATTGAAGTACCAACAGAACCAAGTACACCAGAA
AAGCCAACAGATCCAACACCGCCAATTGAGCCACCTGTAGACCCTGTAGAGCCACCTATTACACCAACGGAGCCA
ACAGAACCGACAGAGCCGACAACACCAACAGAACCTGAAAAACCAGTAGTTCCAACGGAACCGACAACACCAACA
GAACCGACAACTCCTACAGAGCCAAGTGAACCAGAACAACCAACGGAGCCAAGTAAACCAGTAGAACCTGAAAAA
CCAGTTACACCAAGCAAACCAGCAGAACCCGAAAAACCTGTGACACCAACTAAACCAACAGAACCTGAAAAACCA
GTACAACCAGCAGAACCAAGCAAGCCAATCGACGTTGTTGTAACGCCAACAGGGGAATTAAATCACGCTGGAAAT
GGTACACAACAGCCAACAGTCCCTATTGAAACAAGTAATTTGGCAGAAATCACACACGTGCCAAGTGTAACAACA
CCTATTACAACTACAGACGGAGAAAACATTGTAGCTGTAGAAAAAGGTGTTCCACTTACACAAACAGCAGAAGGG
TTAAAACCTATTCAATCGAGTTACAAAGTATTGCCTAGCGGAAATGTAGAAGTAAAAGGTAAGGACGGTAAAATG
AAGGTTTTACCATACACA
Construct 20: EFC0034-1
(SEQ ID NO 20)
AATAAAAAAACACCAAAACCATTAACCCCACCCAAAGCCTTTTTTAACTGGTGTACTGCACAAATACCCACGTAT
GAATGGCAAAACAAAAAGGAGACAATTCTTGCGAGTTCTCGCAAGAATTGCCCTATCATAAAAAAACGATTAACA
AAATACTCACGATTATCGTTTCCAACGAAATTTTACTCCTTTGGAATCATTCTCGTTCGTGCAAAACGAATTGAA
ATTCAAACCCATAGTTATTGGCAGACGATTACAGATGGAATAGAAAACCTTATCTATGAGCCGTCAAACCTTGAA
CGGTTTTCAAACGATACCCATGTTAAAGCCCATTATGAGAACGGACATTGGCATGAGGGCTTGTTAGCAAATTAT
GGTTTTATGAGTAGTGCTTATACAAATACAGTATTCTATCCAAATAACTGGCAAGAAAAACTAACCACTGTATCG
GAATTAAAGTACTTACAGTTACCTGAAATAGAACGTCAAGAACTAGCTCATATTTACAAATACCGAAACGAAATT
GAGTTTTTACAAAAGATAGGTGCCACAACCTTAGCGAATGAAATCATTTTTGACGACTATCGCAACGTTTTTGGA
CTGTATTTTCATAAAGTAGATATGAGAGTAATTACTAAGAAATGGCTAAAAGCGAATAAACAAAAATTAAAAACA
AGAAATCCAACCTTTCATGAATACATGTTAGAAAAAACGTTAAAAGAGCGAAATGCGCCTATGATAAACGAAATA
GAAAAGTACGTGCATTATTCGCAAATTAAGCAACTACCGAAAGAAGTGAACTTAACAAAGTTTCAAAAATGGTTT
ATACGAAAAGGGGAGCGTTTTGACTATTATATGGACTATTTACACATGTTAGAAGAGTTGAACACCCCACTTAAT
AACGATAGTGTATTGTATCCTGAAAACTTACAAGTAGCTCACGATAACGCTATGAACACGCTAAACCTTTTGAAA
AGTGAGATTGAAGAAAAGCAGTATCAAGAACGGAAAAACCAAATTAAAGCGTTAGAAGCTGAAATTGACGATTTG
CTTTTTCTCACACCACATTCATTACAAGAAATCATTCAAGAAGGAAGTATTCTACGCCATTGTGTGGGCAGTCAA
CACTATATCGAACGGCATACGCAAGGAAAAACAACGATTGTTTTTATCCGAAGAAAAGAAAAGCCAGATATGCCT
TACTTCACATTAGAATATCGAAATCAACAGGTTATACAAATACAAGGAAAATGTAACCGTCAGGAAGTACCAGAA
AAGATAAAACAAGCAGTCAGACAATGGCAAGAGAATTTACAGCACGCCTTATCATCA
Protein sequences:
Construct 1: EF0032-2
(SEQ ID NO 21)
MVTGILDDWNYASRSLYSEPYPDLKNLVDQTKKENDTFYRLENLNGVSANDGINYGYSGISMFSSVRNRHSSTYL
NALGFRSRGTNLNIRYQNNTLLMDALMGIKYNIAENNPMKFGFERQAAAGKYQLYRNENALPLGFLADKEIYNVR
QPLNDNLGSQTNLLNALANTNERYFTFYQPTMTLQNNVTITQNTAGVTFTEKQHNVAKEISYTVNVPANTQAYLS
LFPTDFAQLESSTATVTVNGSSQQSQIGITGQYYNLGYYPKDTTVNFKVSFYGTKAVSFVQPQVVGLNTNAFEKA
ISAVQEKGVDLTTGKRSASGTFTADKDQVLVTTIPYDKGWRVKIDGKKVTPKAFKDAFLSVPVSAGTHTIQFSYL
PEGLIPGIVLFVLCTGGFVAYVTLIPARRNRKKEDK
Construct 2: EF0270-2
(SEQ ID NO 22)
AIAAVVGFGLTFFFWKDNTVEEEEVIIDKTTIKKENITSPVKGRVLSLKNAEDPAFANGALGNGVVIEPTEGKVV
APFDGTIVTLFPTKHALGLISDNGTELLIHIGIDTVQLEGEGFEAFVKQGDRVKKGQTLVTFDLEGIKKAGFSTQ
IPIVVTNTADYLDILEVGSNEVSTSDDLLTALI
Construct 3: EF0428
(SEQ ID NO 23)
LPMIIKQEQFIPKDTVETTIDLLIRNLTTIKDNTGEFLLDFDGLKVDDKSWTIWNWPQGVGLYGIYKNYRNTKSE
KALQVVNDWFEGRMQEGAPPKNVNTMAPLLTMAYLYEDTKDSKYIPYLEQWAEWVMEEMPRTNEGGLQHATYGPE
NKNQLWDDTLMMTVLPLAKIGKLLNRLDYLEEAKHQFLIHIKYLQDKKSGLWYHGWTFEGNHNYAEALWARGNCW
ITIAIPEIIEILELPKGDSLREFLLSTLNAQVAALAKYQDESGLWHTLINDSNSYLESSATAGFAYGILKAVHKK
YISSEYEEVANKAIAGLLNEIDETGEVQHVSVGTGMGDNLDFYRTIGMTAMPYGQSLTILCLTELLVSYC
Construct 4: EF0779-2
(SEQ ID NO 24)
GYIIFNAVYLTGLLESKPLIISHRGVTNSNGVQNTIPAMERTIKFKPDYIEIDVQETKDHQFVVMHDANLQELAG
VDGTPQEFTLAELTKMTVKENGQEAPIASFDDYLAKANQAKQKLLVEIKTSKQDSQGALSNFIEKYERPLIKNNH
QVQSLDYNVIKAFKKAKSKVKVSFILPYNFTFPETQADLYTMEATTLNDTFILKADQQKKAVYAWTVNDSEVLSK
MLFMDVAGVITDDLELVNEEVNDFEKNPSYADRILHYIFMLPSVASQ
Construct 5: EF0792-1
(SEQ ID NO 25)
LAQGNRLAVDQWQANQVVLSKEANSNLNVSVLDENVKETISGGKIAPIGQQSLAIRPADDKKAELTNVSLFGIEK
ESFLMPKVIEGNAFTDKNQVIASETLKNQGFKIGDKLTAGKYDEQLEIVGFISKSSYNIVPVIYTSLDTWRSIKY
GDNPAMAKMVNGFIVRSKDNTEVKTTNKDSQ
Construct 6: EF1026
(SEQ ID NO 26)
MKKEIIVYTISDSLGETSQKLLAAASAQYPDISFLNRYNFSFVTTEEELLEILKDALKDKALVVSTLVSKQLITA
AKEFSERTGLLYLDLMAPFFELIQAKAGVDPIEEPGRRHQLDRAYFDKISAIEFAVKYDDGKNPQGFLDSDILLL
GVSRTSKTPVSMYLANQGYRVSNLPLIPEVPLPPILEEMDPQKMIGLVCSPETLGQIRSSRLASLGLGNETSYTN
VERIEQELAYAEEIFAKYGIPVIDVTAKSVEETAFLIKEKLDERN
Construct 7: EF1032 IB
(SEQ ID NO 27)
MEWIEIKHATQNNLKNISVNIPKKQLTVVTGLSGSGKSSLVFDTLAAESRRELNDTFSSFVQNYLPKYGRPEVEK
IENLPVAIVIDQKKVAGNSRSTVGTYTDIYTFLRLLFSRAGSPFVGYSDTFSFNHPDGKCPTCDGLGKITEINLH
QLVDYDKSLNKGPIDFPTFTVGNWRWKRYAHSGLFDLDKKIKDYSPEELALFLYAPQQKLANPPKEWPHTALYEG
IVPRMQRSILHTDEGKRHQKYLNHFVTVKRCPDCLGSRVNERVRSCKINQKSIADAVDMPLTELHSFIRSMDLSL
IKTIQEELLVRLEALINIGLSYLTLGRATETLSGGEAQRIKIAKYVNSALNDIMYILDEPSAGLHPKDIERISRA
LLNLKNKGNTVVLVEHNPQLIREADFIIDIGPFAGENGGHVQFSGTYDAFLVSKTLTSQALQEPLPLNDQPRKAR
KSLSIEHATLHNLNNLSVEVPLGVLTVICGVAGSGKSSLAEEIYQKAQADNQEIIHLSQKSITANLRSTPMTYLN
IFDKVRKLFAEENHVSPALFSYNSKGACPTCKGKGIIVSDMSFMEDVTSICETCHGTRYKEEVLHYLYNGKNIVE
VLALSVKDGYDFFKDQPFALSLKNLLEVGLSYLKLNQSLSTLSGGELQRVKLADTLHQKKAIYLMDEPTDGLHLI
DIQQSLQLFNRMVEEGNSLILLEHHIDVIKSADWLIELGPEGGKNGGQLLFTGTPANMLNSTHSITKGYL
Construct 8: EF1060-1
(SEQ ID NO 28)
DSNKAAEQKIAISSEAAISTMEPHTAGDTTSTLVMNQVYEGLYVLGKEDELELGVAAEEPAISEDETVYTFKIRE
DAKWSNDDPVTANDFVYAWQQVASPKSGSIHQALFFDVIKNAKEIALEGADVNTLGVKALDDKTLEITLERPTPY
LKSLLSFPVLFPQNEKYIKEQGDKYATDAEHLIYNGPFKLKEWDNASSDDWTYEKNDTYWDAEKVKLTEAKVSVI
KSPTTAVNLFDSNELDVVNKLSGEFIPGYVDNPAFLSIPQFVTYFLKMNSVRDGKENPALANNNIRKALAQAFDK
ESFVKEVLQDQSTATDQVIPPGQTIAPDGTDFTKLAAKKNNYLTYDTAKAKEFWEKGKKEIGLDKIKLEFLTDDT
DSAKKAAEFFQFQLEENLDGLEVNVTQVPFTIRVDRDQTRDYDLELSGWGTDYRDPLTVMRIFTSDSTLGGVTFK
SDTYDQLIQETRTTHAADQEARLNDFAQAQDILVNQETVLAPIYNRSISVLANQKIKDLYWHSFGPTYSLKWAYV
N
Construct 9: EF1093-1
(SEQ ID NO 29)
EENGESAQLVIHKKKMTDLPDPLIQNSGKEMSEFDKYQGLADVTFSIYNVTNEFYEQRAAGASVDAAKQAVQSLT
PGKPVAQGTTDANGNVTVQLPKKQNGKDAVYTIKEEPKEGVVAATNMVVAFPVYEMIKQTDGSYKYGTEELAVVH
IYPKNVVANDGSLKKVGTAENEGLNGAEFVISKSEGSPGTVKYIQGVKDGLYTWTTDKEQAKRFITGKSYEIGEN
DFTEAENGTGELTVKNLEVGSYILEEVKAPNNAELIENQTKTPFTIEANNQTPVEKTVKNDTSKVDKTTPSLDGK
DVAIGEKIKYQISVNIPLGIADKEGDANKYVKFNLVDKHDAALTFDNVTSGEYAYALYDGDTVIAPENYQVTEQA
NGFTVAVNPAYIPTLTPGGTLKFVYFMHLNEKADPTKGFKNEANVDNGHTDDQTPPTVEVVTGGKRFIKVDGDVT
ATQALAGASFVVRDQNSDTANYLKIDETTKAATWVKTKAEATTFTTTADGLVDITGLKYGTYYLEETVAPDDYVL
LTNRIEFVVNEQSYGTTENLVSPEKVPNKHKGTLPST
Construct 10: EF1277
(SEQ ID NO 30)
MNIFAVRLKEALTAKNIKPSDLAKKTGIGKSSISDWLAGRYEAKQDKVYRIADALDINEAWLMGQEVPMEKNAST
IDRIYKKLEPQRQAIVYQFAEQQLHEQQTQAEILSFPRRDEMTLAAHAGDPEKIFSKEEIEKIHDYLDEIDAKYQ
QSISSDKKED
Construct 11: EF1355
(SEQ ID NO 31)
MAYQFKLPDIGEGIAEGEIVKWFVKPGDTINEDDTLLEVQNDKSVEEIPSPVTGTVKNIVVPEGTVANVGDVLIE
IDAPGHEDNDAAPAAPAQEQTPAQPAAVPTTEAAGGFFQFKLPDIGEGIAEGEIVKWFVKAGDTINEDDSLLEVQ
NDKSVEEIPSPVTGTVKNIVVPEGTVANVGDVLVEIDAPGHNSAAPSVAAPATDAPKAEASAPAASTGVVAAADP
NKRVLAMPSVRQYAREKDVDITQVTATGKGGRVIKADIDAFVSGGSQAAPATEAAATEAAPKAEAAAPKAAPKAF
TSDLGEMETREKMTPTRKAIAKAMVNSKHTAPHVTLHDEVEVSKLWDHRKKFKDVAAANGTKLTFLPYVVKALTS
TVQKFPILNASIDDAAQEIVYKNYFNIGIATDTDHGLYVPNVKNANTKSMFAIADEINEKAALAIEGKLTAQDMR
DGTITISNIGSVGGGWFTPVINYPEVAILGVGTIAQEPVVNADGEIVVGRMMKLSLSFDHRIVDGATAQKAMNNI
KRLLADPELLLMEG
Construct 12: EF1692
(SEQ ID NO 32)
MAKRIIIMNFDIESKSYQAFSEIKKMQAERQLKGEQMAVVTHVNDGQHQFKINDFIDFTGNNHTSKDSMIGMLVG
ILGGPLGILFGWFAGSMYGASKDAKEIQEAQTVFEHVIQKIDEGQTGLLLIAEEEDNRPLNQLVMFDLGGEITRL
DLEEVQQEINDANEVANEAKQSWQAKKEQHKEATSKEE
Construct 13: EF2703-1
(SEQ ID NO 33)
GSYLKKTIDIGYVPIKNDYNEAQNKDSQSFLIMGLDNTIERKLGTTRTDAMMVITVNNKTKKITYLSLPRDSFVQ
IDAKNYQGMQRIEAAYTYDGPTASVNTVEKLLNIPINHYVVFNFLSFIKLIDAVGGIDVNVKQAFDGVTKDGPGS
IHFDAGKQHLDGTKALSYARERHSDNDIMRGFRQQEIIQAVEDKLKSGQSIMKIMDIIDSLNGNIQTDVDSNELT
HLVKEGLTWTNYDKQQLSFDWRTFSNEGRSMVELYPDSIENVRHQLRVSLNLEKPDERDQDGYVFHTNGEFLYQS
DYTVQDEAAEENEMTSINGNTYIGVPGNTQTGPLPSVKTENGFIK
Construct 14: EF3051-1
(SEQ ID NO 34)
FSSWFDDSYVKASAESSKTKEPAPVKIEKKVKPLSYGQQVNQEIEKKQYDGHLDLPLELQTDAKWKDTAYGFGNV
DKPNTIEINGCAIVSLAMVGSYMDHQEVTPLDVLAWAKNDFFMEGQGTAWSIFSAYAEMKGYNCQEIGDIETVAA
FLKEGHPVIISVKPGYFTTTGHIMVMSGVDEKGDFWINDPNDSEEKGHSKRTFTAEEVMNEALNFWAFY
Construct 15: EF3177
(SEQ ID NO 35)
MDLHLTVDLKKTKTLIDLADQAHLIIEQTTKLPKKEAAILFLLIDNQLVALGLAEEKATYKEVSFDHSILLKPTW
DTELAYLAQAFLDDAKESGLTLEATPTTVKIPKSAVATVTNYKETVTFILERFGYSLFKKPAPKKARPAKARHKW
TKEVSQIPFYIDTRQSKATVFWQKRNEMLIKAGATMMPEAPLNKDGSVGFSARFGEKLRDERKGQFKDFVTTEDI
VLKSVNEVGLFLYFAGTNSWLELVDENGKTLNEWTVVE
Construct 16: EF3183-1
(SEQ ID NO 36)
SEFNFAVTPTIPENQVDKSKTYFDLKMAPGAKQTVEIQLRNDTDEDITIENTVNSATTNLNGVVEYGQNGIKPDK
TLRFNLKDYVEAPKEIILPKHSQKTLPLTITMPKDSFDGVMAGGITLKEKKKETTTSADQSKGLAINNEYSYVVA
IILQQNETKVQPDLKLLGVKPGQVNARNVINVSLQNPQAAYLNQLHLINTVSKGGETLYQSDTEDMQVAPNSNFS
YPISLKGERLTPGKYVLKSTAYGVKDEKGTYQVKGANGEERYLYKWEFTKEFTISGDVAKELNEKDVTIKGTNWW
LYLLIALIILALLLLIFFLYRKKKKEEEQQSEQ
Construct 17: EF3207
(SEQ ID NO37)
MSKNFWATLPKPFFVLAPMEDVTDVVFRHVVKEAGAPDVLFTEFTNSDSYCHPEGKDSVRGRLVFTEDEQPMVAH
IWGDKPEFFREMSIGVAEMGFQGLDINMGCPVPNVAERGKGSGLILRPEVAAELIDAAKAGGLPVSVKTRIGFTE
MAEMEAWITHLLEQDIANLSIHLRTRKEMSKVDAHWEVIPQIMAIRDRVAPQTTITINGDIPDRQKGLELAEQYG
VDGIMIGRGIFKNPYAFEKEPKTHTPQELLGLLRLQLDLQDKYAELVPRSIVGLHRFFKIYVKGFPGASDLRAQL
MNTKSTDEVRQLLATFETEHGVLD
Construct 18: EF3256-1
(SEQ ID NO 38)
ADKKDNTTNSSSAASSETKKSTESSAPAKKVAGGDLKDGTYKLEEKNEKNGYRAVFEMTVKDGKITESKYDNINA
DGKSKTEDTKYEESMKAKSGVGPKEYIKQLNDSFVKAQSASGVEVVTGATHSSESFQNYAQQLIQAAQAGNTDTI
EIDNGATLKDGTYSLKEKNDSNGYHTTFSMTVKDGKVTESNYDNVNADGKSKKDDTEYESKMEDVAGVGPKEYIE
TLNKEFVKAMGEEDGSPAGVEVVTGATHSTHSFINYAQQLVNAAEKGDTTEIVVDNIVTK
Construct 19: EFA0042-1
(SEQ ID NO 39)
FAQEIIPDDTTTPPIEVPTEPSTPEKPTDPTPPIEPPVDPVEPPITPTEPTEPTEPTTPTEPEKPVVPTEPTTPT
EPTTPTEPSEPEQPTEPSKPVEPEKPVTPSKPAEPEKPVTPTKPTEPEKPVQPAEPSKPIDVVVTPTGELNHAGN
GTQQPTVPIETSNLAEITHVPSVTTPITTTDGENIVAVEKGVPLTQTAEGLKPIQSSYKVLPSGNVEVKGKDGKM
KVLPYT
Construct 20: EFC0034-1
(SEQ ID NO 40)
NKKTPKPLTPPKAFFNWCTAQIPTYEWQNKKETILASSRKNCPIIKKRLTKYSRLSFPTKFYSFGIILVRAKRIE
IQTHSYWQTITDGIENLIYEPSNLERFSNDTHVKAHYENGHWHEGLLANYGFMSSAYTNTVFYPNNWQEKLTTVS
ELKYLQLPEIERQELAHIYKYRNEIEFLQKIGATTLANEIIFDDYRNVFGLYFHKVDMRVITKKWLKANKQKLKT
RNPTFHEYMLEKTLKERNAPMINEIEKYVHYSQIKQLPKEVNLTKFQKWFIRKGERFDYYMDYLHMLEELNTPLN
NDSVLYPENLQVAHDNAMNTLNLLKSEIEEKQYQERKNQIKALEAEIDDLLFLTPHSLQEIIQEGSILRHCVGSQ
HYIERHTQGKTTIVFIRRKEKPDMPYFTLEYRNQQVIQIQGKCNRQEVPEKIKQAVRQWQENLQHALSS

### Example 2

### Conservation of Selected Vaccine Antigen-Encoding ORFs in E. Faecalis Clinical Isolates

### Experimental Procedures:

### Bacterial Culture and Isolation of Genomic DNA

Seven ml Luria Broth medium was inoculated with E. faecalis cells from frozen stabs, and cells were grown under aerobic conditions at 37° C., overnight. The cultures were harvested by centrifugation at 16,000×g for 15 mM and the supernatants were removed. Genomic DNA was purified using the Wizard genomic DNA purification kit (Promega) according to the manufacturer's instructions. Briefly, bacterial pellets were resuspended in 480 μl 50 mM EDTA and 120 μl lysozyme solution (10 mg/ml) (Sigma), incubated 60 min at 37° C., solutions were centrifuged at 16,000×g for 2 min, and the supernatants were removed. The cell pellets were resuspended in 600 μl Nuclei Lysis Solution (Wizard genomic DNA purification kit, Promega), incubated for 5 min at 80° C., cooled to room temperature, 3 μl RNase solution added (4 mg/ml, Promega), and incubated for 1 hr at 37° C. After cooling to 4° C. on ice, 200 μl Protein Precipitation Solution (Wizard genomic DNA purification kit, Promega) was added, the suspensions were vortexed, incubated on ice for 5 min, and centrifuged at 16,000×g for 5 min. The supernatants were transferred to new tubes containing 600 μl of room temperature isopropanol. After mixing, the solutions were centrifuged at 16,000×g for 2 m, the precipitated DNA was gently washed with 600 μl of room temperature 70% (v/v) ethanol, air-dried for 15 min at room temperature, and dissolved in 60 μl ddH₂O for 1 hr at 65° C.

### PCR:

Primers were designed for the nine ORFs; EF2703-1, EF0032-2, EF3177, EF0792-1, EF1277, EF3256-1, EF0270-2, EF1060-1, and EF3207 (Table 3) by multiple alignment of several Enterococcus spp. sequences (performed using the program VectorNTI). These nine E. faecalis ORFs were selected for PCR analysis as the recombinant protein antigens encoded by these ORFs showed the highest level of protection (≧60-100%) in the rat endocarditis model (Table 3). In order to facilitate PCR amplification, the optimal fragment size was set to 800 bp. PCR was performed in reaction volumes of 30 μl, containing 1 U Taq polymerase (Solis Biodyne, Estonia), 0.2 mM dNTPs (Invitrogen), 0.4 μM of each oligonucleotide (MWG, Germany), 2 mM MgCh and 1× buffer (Solis Biodyne, Estonia). As standard, 30 cycles (1×: 5 mM at 95° C.; 30×: 30 sec at 95° C., 30 sec at 55° C., 1 min 40 sec at 72° C.; 1×5 min at 72° C.) were performed, unless it was necessary to adapt conditions for individual primer pairs. PCR amplification was performed in a Biometra T3 Thermocycler.

In order to avoid false-negative results, all PCR reactions that were negative in the first amplification round were repeated. Optimization of PCR conditions was performed to successfully amplify the target sequence. Optimizations included increasing or lowering of the annealing temperature by 2 or 5° C., addition of betaine (final concentration 1M), or addition of DMSO (dimethylsulfoxide) to a final concentration of 2% (v/v). Touch-down-PCR conditions were used in cases where difficulties were encountered with target sequence amplification. The PCR fragments were subsequently visualized by electrophoresis on a 1% (w/v) agarose gel and staining with ethidium bromide (1:10000).

### Clinical Isolates:

Details of all isolates of E. faecalis used for PCR and sequencing are summarized in Table 4. The 46 E. faecalis isolates examined were selected to represent the serotypes that are most frequently isolated from patients. For further reference see Maekawa, S. et al., Microbiol Immunol. 1992; 36(7):671-681 or Shankar, V. et al., Infect Immun. 1999; 67(1):193-200.

### DNA Sequencing:

The oligonucleotides used for sequencing were the same as those used for PCR amplification of the nine selected E. faecalis ORFs. Sequencing was performed by Agowa Biotech AG, Berlin, Germany.

### Results

PCR was performed from genomic DNA isolated from a series of 46 independent E. faecalis clinical isolates using primers specific for the nine selected ORFs. All PCR amplicons obtained were sequenced. The results of the sequencing are presented in Table 5, where the determined DNA sequences together with the corresponding translated protein sequences are summarized.

TABLE 4
E. faecalis strains utilized for PCR and sequencing of vaccine-antigen
encoding ORFs

| | | Origin | |
|---|---|---|---|
| Strain | Species | (reference or source) | Country |
| S1 | E. faecalis | Clinical isolate | Japan |
| | | (Maekawa et al., 1992) | |
| S2 | E. faecalis | Clinical isolate | Japan |
| | | (Maekawa et al., 1992) | |
| S3 | E. faecalis | Clinical isolate | Japan |
| | | (Maekawa et al., 1992) | |
| S4 | E. faecalis | Clinical isolate | Japan |
| | | (Maekawa et al., 1992) | |
| S5 | E. faecalis | Clinical isolate | Japan |
| | | (Maekawa et al., 1992) | |
| S6 | E. faecalis | Clinical isolate | Japan |
| | | (Maekawa et al., 1992) | |
| S7 | E. faecalis | Clinical isolate | Japan |
| | | (Maekawa et al., 1992) | |
| S8 | E. faecalis | Clinical isolate | Japan |
| | | (Maekawa et al., 1992) | |
| S9 | E. faecalis | Clinical isolate | Japan |
| | | (Maekawa et al., 1992) | |
| S10 | E. faecalis | Clinical isolate | Japan |
| | | (Maekawa et al., 1992) | |
| S11 | E. faecalis | Clinical isolate | Japan |
| | | (Maekawa et al., 1992) | |
| S12 | E. faecalis | Clinical isolate | Japan |
| | | (Maekawa et al., 1992) | |
| S13 | E. faecalis | Clinical isolate | Japan |
| | | (Maekawa et al., 1992) | |
| S14 | E. faecalis | Clinical isolate | Japan |
| | | (Maekawa et al., 1992) | |
| S15 | E. faecalis | Clinical isolate | Japan |
| | | (Maekawa et al., 1992) | |
| S16 | E. faecalis | Clinical isolate | Japan |
| | | (Maekawa et al., 1992) | |
| S17 | E. faecalis | Clinical isolate | Japan |
| | | (Maekawa et al., 1992) | |
| S18 | E. faecalis | Clinical isolate | Japan |
| | | (Maekawa et al., 1992) | |
| S19 | E. faecalis | Clinical isolate | Japan |
| | | (Maekawa et al., 1992) | |
| S20 | E. faecalis | Clinical isolate | Japan |
| | | (Maekawa et al., 1992) | |
| S21 | E. faecalis | Clinical isolate | Japan |
| | | (Maekawa et al., 1992) | |
| 12030 (S22) | E. faecalis | Clinical isolate | Germany |
| 12107 (S23) | E. faecalis | Clinical isolate | Germany |
| OG1RF (S24) | E. faecalis | Laboratory strain | Germany |
| S25 | E. faecalis | Clinical isolate | Germany |
| S26 | E. faecalis | Clinical isolate | USA |
| | | (Shankar et al., 1999) | |
| HSM3143a | E. faecalis | Clinical isolate | Portugal |
| HSM3686 | E. faecalis | Clinical isolate | Portugal |
| HSM3720 | E. faecalis | Clinical isolate | Portugal |
| HSM3840 | E. faecalis | Clinical isolate | Portugal |
| HSM4182 | E. faecalis | Clinical isolate | Portugal |
| U-1482 | E. faecalis | Clinical isolate | Portugal |
| U-1740 | E. faecalis | Clinical isolate | Portugal |
| U-1780 | E. faecalis | Clinical isolate | Portugal |
| U-1881 | E. faecalis | Clinical isolate | Portugal |
| U-2265 | E. faecalis | Clinical isolate | Portugal |
| 32 | Enterococcus sp. | Skin soft tissue infection | Germany |
| 88 | Enterococcus sp. | Skin soft tissue infection | Germany |
| 99 | Enterococcus sp. | Skin soft tissue infection | Germany |
| 44 | Enterococcus sp. | Skin soft tissue infection | Germany |
| 4 | Enterococcus sp. | Urinary tract infection | Germany |
| 100 | Enterococcus sp. | Urinary tract infection | Germany |
| 2 | Enterococcus sp. | Urinary tract infection | Germany |
| 31 | Enterococcus sp. | Intra-abdominal infection | Germany |
| 75 | Enterococcus sp. | Intra-abdominal infection | Germany |
| 64 | Enterococcus sp. | Intra-abdominal infection | Germany |

TABLE 5
Summary on the sequence conservation of the selected 9 E. faecalis genes in
forty-six isolates. In case a particular sequence (or fragment) has been determined, then
the respective SEQ ID NOs are shown, first the encoding DNA, then the translated
polypeptide sequence as second.

| Strain | EF0032 | EF0270 | EF0792 | EF1060 | EF1277 | EF2703 | EF3177 | EF3207 | EF3256 |
|---|---|---|---|---|---|---|---|---|---|
| S1 | 85/86 | 179/ | 271/ | 355/ | 441/ | 513/ | 605/ | n.d. | 713/ |
| | | 180 | 272 | 356 | 442 | 514 | 606 | | 714 |
| S2 | 87/88 | 181/ | 273/ | 357/ | n.d. | 515/ | 607/ | n.d. | 715/ |
| | | 182 | 274 | 358 | | 516 | 608 | | 716 |
| S3 | 89/90 | 183/ | 275/ | 359/ | n.d. | 517/ | 609/ | n.d. | 717/ |
| | | 184 | 276 | 360 | | 518 | 610 | | 718 |
| S4 | 91/92 | 185/ | 277/ | 361/ | 443/ | 519/ | 611/ | 691/ | 719/ |
| | | 186 | 278 | 362 | 444 | 520 | 612 | 692 | 720 |
| S5 | 93/94 | 187/ | 279/ | 363/ | 445/ | 521/ | 613/ | n.d. | 721/ |
| | | 188 | 280 | 364 | 446 | 522 | 614 | | 722 |
| S6 | 95/96 | 189/ | 281/ | 365/ | n.d. | 523/ | 615/ | n.d. | 723/ |
| | | 190 | 282 | 366 | | 524 | 616 | | 724 |
| S7 | 97/98 | 191/ | 283/ | 367/ | 447/ | 525/ | 617/ | n.d. | 725/ |
| | | 192 | 284 | 368 | 448 | 526 | 618 | | 726 |
| S8 | 99/100 | 193/ | 285/ | 369/ | 449/ | 527/ | 619/ | n.d. | 727/ |
| | | 194 | 286 | 370 | 450 | 528 | 620 | | 728 |
| S9 | 101/ | 195/ | 287/ | 371/ | n.d. | 529/ | 621/ | n.d. | 729/ |
| | 102 | 196 | 288 | 372 | | 530 | 622 | | 730 |
| S10 | 103/ | 197/ | 289/ | 373/ | n.d. | 531/ | 623/ | n.d. | 731/ |
| | 104 | 198 | 290 | 374 | | 532 | 624 | | 732 |
| S11 | 105/ | 199/ | n.d. | 375/ | 451/ | 533/ | 625/ | n.d. | 733/ |
| | 106 | 200 | | 376 | 452 | 534 | 626 | | 734 |
| S12 | 107/ | 201/ | 291/ | 377/ | 453/ | 535/ | n.d. | n.d. | 735/ |
| | 108 | 202 | 292 | 378 | 454 | 536 | | | 736 |
| S13 | 109/ | 203/ | 293/ | 379/ | n.d. | 537/ | 627/ | n.d. | 737/ |
| | 110 | 204 | 294 | 380 | | 538 | 628 | | 738 |
| S14 | 111/ | 205/ | 295/ | 381/ | 455/ | 539/ | 629/ | n.d. | 739/ |
| | 112 | 206 | 296 | 382 | 456 | 540 | 630 | | 740 |
| S15 | 113/ | 207/ | 297/ | 383/ | 457/ | 541/ | 631/ | n.d. | 741/ |
| | 114 | 208 | 298 | 384 | 458 | 542 | 632 | | 742 |
| S16 | 115/ | 209/ | 299/ | 385/ | 459/ | 543/ | 633/ | n.d. | 743/ |
| | 116 | 210 | 300 | 386 | 460 | 544 | 634 | | 744 |
| S17 | 117/ | 211/ | n.d. | n.d. | 461/ | 545/ | 635/ | n.d. | 745/ |
| | 118 | 212 | | | 462 | 546 | 636 | | 746 |
| S18 | 119/ | 213/ | 301/ | 387/ | 463/ | 547/ | 637/ | n.d. | 747/ |
| | 120 | 214 | 302 | 388 | 464 | 548 | 638 | | 748 |
| S19 | 121/ | 215/ | 303/ | 389/ | 465/ | 549/ | 639/ | n.d. | 749/ |
| | 122 | 216 | 304 | 390 | 466 | 550 | 640 | | 750 |
| S20 | 123/ | 217/ | 305/ | 391/ | 467/ | 551/ | 641/ | n.d. | 751/ |
| | 124 | 218 | 306 | 392 | 468 | 552 | 642 | | 752 |
| S21 | 125/ | 219/ | 307/ | 393/ | n.d. | 553/ | 643/ | n.d. | n.d. |
| | 126 | 220 | 308 | 394 | | 554 | 644 | | |
| 12030 | 127/ | 221/ | 309/ | 395/ | 469/ | 555/ | 645/ | n.d. | 753/ |
| (S22) | 128 | 222 | 310 | 396 | 470 | 556 | 646 | | 754 |
| 12107 | 129/ | 223/ | 311/ | 397/ | 471/ | 557/ | 647/ | n.d. | 755/ |
| (S23) | 130 | 224 | 312 | 398 | 472 | 558 | 648 | | 756 |
| OG1RF | 131/ | 225/ | 313/ | 399/ | 473/ | 559/ | 649/ | n.d. | 757/ |
| (S24) | 132 | 226 | 314 | 400 | 474 | 560 | 650 | | 758 |
| S25 | 133/ | 227/ | 315/ | 401/ | 475/ | 561/ | 651/ | n.d. | 759/ |
| | 134 | 228 | 316 | 402 | 476 | 562 | 652 | | 760 |
| S26 | 135/ | 229/ | n.d. | 403/ | 477/ | 563/ | 653/ | 693/ | 761/ |
| | 136 | 230 | | 404 | 478 | 564 | 654 | 694 | 762 |
| HSM3143a | 137/ | 231/ | 317/ | 405/ | 479/ | 565/ | 655/ | n.d. | 763/ |
| | 138 | 232 | 318 | 406 | 480 | 566 | 656 | | 764 |
| HSM3686 | 139/ | 233/ | 319/ | 407/ | 481/ | 567/ | 657/ | n.d. | 765/ |
| | 140 | 234 | 320 | 408 | 482 | 568 | 658 | | 766 |
| HSM3720 | 141/ | 235/ | 321/ | 409/ | n.d. | 569/ | 659/ | 695/ | 767/ |
| | 142 | 236 | 322 | 410 | | 570 | 660 | 696 | 768 |
| HSM3840 | 143, 145/ | 237/ | 323/ | 411/ | 483/ | 571/ | 661/ | 697/ | 769/ |
| | 144, 146 | 238 | 324 | 412 | 484 | 572 | 662 | 698 | 770 |
| HSM4182 | 147/ | 239/ | 325/ | 413/ | n.d. | 573/ | 663/ | 699/ | 771/ |
| | 148 | 240 | 326 | 414 | | 574 | 664 | 700 | 772 |
| U-1482 | 149/ | 241/ | 327/ | 415/ | 485/ | 575/ | n.d. | n.d. | 773/ |
| | 150 | 242 | 328 | 416 | 486 | 576 | | | 774 |
| U-1740 | 151/ | 243/ | 329/ | 417/ | 487/ | 577/ | 665/ | n.d. | 775/ |
| | 152 | 244 | 330 | 418 | 488 | 578 | 666 | | 776 |
| U-1780 | 153/ | 245/ | n.d. | 419/ | 489/ | 579/ | 667/ | n.d. | n.d. |
| | 154 | 246 | | 420 | 490 | 580 | 668 | | |
| U-1881 | 155/ | 247/ | 331/ | 421/ | n.d. | 581/ | 669/ | n.d. | 777/ |
| | 156 | 248 | 332 | 422 | | 582 | 670 | | 778 |
| U-2265 | 157/ | 249/ | 333/ | n.d. | 491/ | 583/ | 671/ | n.d. | 779/ |
| | 158 | 250 | 334 | | 492 | 584 | 672 | | 780 |
| 32 | 159/ | 251/ | 335/ | n.d. | 493/ | 585/ | 673/ | 701/ | 781/ |
| | 160 | 252 | 336 | | 494 | 586 | 674 | 702 | 782 |
| 88 | 161/ | 253/ | 337/ | 423/ | 495/ | 587/ | n.d. | n.d. | 783/ |
| | 162 | 254 | 338 | 424 | 496 | 588 | | | 784 |
| 99 | 163/ | 255/ | 339/ | 425/ | 497/ | 589/ | 675/ | n.d. | 785/ |
| | 164 | 256 | 340 | 426 | 498 | 590 | 676 | | 786 |
| 44 | 165/ | 257/ | 341/ | 427/ | 499/ | 591/ | 677/ | 703/ | 787/ |
| | 166 | 258 | 342 | 428 | 500 | 592 | 678 | 704 | 788 |
| 4 | 167/ | 259/ | 343/ | 429/ | 501/ | 593/ | 679/ | n.d. | 789/ |
| | 168 | 260 | 344 | 430 | 502 | 594 | 680 | | 790 |
| 100 | 169/ | 261/ | 345/ | 431/ | 503/ | 595/ | 681/ | n.d. | n.d. |
| | 170 | 262 | 346 | 432 | 504 | 596 | 682 | | |
| 2 | 171/ | 263/ | 347/ | 433/ | 505/ | 597/ | 683/ | 705/ | 791/ |
| | 172 | 264 | 348 | 434 | 506 | 598 | 684 | 706 | 792 |
| 31 | 173/ | 265/ | 349/ | 435/ | 507/ | 599/ | 685/ | 707/ | 793/ |
| | 174 | 266 | 350 | 436 | 508 | 600 | 686 | 708 | 794 |
| 75 | 175/ | 267/ | 351/ | 437/ | 509/ | 601/ | 687/ | 709/ | 795/ |
| | 176 | 268 | 352 | 438 | 510 | 602 | 688 | 710 | 796 |
| 64 | 177/ | 269/ | 353/ | 439/ | 511/ | 603/ | 689/ | 711/ | 797/ |
| | 178 | 270 | 354 | 440 | 512 | 604 | 690 | 712 | 798 |

“n.d.” means “not determined”.

1. A protective peptide consisting of an antigen of E. faecalis of the SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40 or a functional active variant of the antigen of any of the SEQ ID NOs: 21 to 40.

2. A protective peptide consisting of an antigen of E. faecalis of the SEQ ID NO: 21, SEQ ID NO: 22, SEQ ID NO: 24 or SEQ ID NO: 25 or a functional active variant of an antigen of E. faecalis of the SEQ ID NO: 21, SEQ ID NO: 22, SEQ ID NO: 24 or SEQ ID NO: 25, and
a) 1 to 450 additional amino acid residue(s), preferably 1 to 400, 1 to 350, 1 to 300, 1 to 250, 1 to 200, 1 to 150, more preferably 1 to 100, even more preferably at most 1 to 50, most preferably 1, 2, 3, 4, 5, 10, 20, 30 or 40 additional amino acids residue(s) if the antigen is SEQ ID NO: 21, wherein the additional amino acid residue(s) is/are flanking the antigen C-terminally, N-terminally or C- and N-terminally; or
b) 1 to 400 additional amino acid residue(s), preferably 1 to 350, 1 to 300, 1 to 250, 1 to 200, 1 to 150, more preferably 1 to 100, even more preferably at most 1 to 50, most preferably 1, 2, 3, 4, 5, 10, 20, 30 or 40 additional amino acids residue(s) if the antigen is SEQ ID NO: 22, wherein the additional amino acid residue(s) is/are flanking the antigen C-terminally, N-terminally or C- and N-terminally; or
c) 1 to 300 additional amino acid residue(s), preferably 1 to 250, 1 to 200, 1 to 150, more preferably 1 to 100, even more preferably at most 1 to 50, most preferably 1, 2, 3, 4, 5, 10, 20, 30 or 40 additional amino acids residue(s) if the antigen is SEQ ID NO: 24, wherein the additional amino acid residue(s) is/are flanking the antigen C-terminally, N-terminally or C- and N-terminally; or
d) 1 to 125 additional amino acid residue(s), preferably 1 to 100, 1 to 80, 1 to 60, more preferably 1 to 40, even more preferably at most 1 to 30, most preferably 1, 2, 3, 4, 5, 10, 15, 20, or 25 additional amino acids residue(s) if the antigen is SEQ ID NO: 25, wherein the additional amino acid residue(s) is/are flanking the antigen C-terminally, N-terminally or C- and N-terminally.

3. The protective peptide of claim 1 further consisting of at least one amino acid residue heterologous to the antigen, preferably an additional amino acid sequence comprising a marker protein.

4. A protective peptide, characterized in that comprises at least 2, preferably at least 3, more preferably at least 4 antigens as defined in claim 1.

5. The protective peptide of claim 2 wherein the additional amino acid residue(s) consist of at least one amino acid residue heterologous to the antigen, preferably an additional amino acid sequence comprising a marker protein.

6. The protective peptide of claim 1 wherein the functional active variant is essentially identical to any of the antigens of the SEQ ID NOs: 21 to 40, but differs from the antigens of any of the SEQ ID NOs: 21 to 40 in that it is derived from a homologous sequence of a different serotype of E. faecalis.

7. The protective peptide of claim 1, wherein the functional active variant is a portion of any of the SEQ ID NOs: 21 to 40 consisting of at least 60%, preferably at least 70%, more preferably at least 80%, still more preferably at least 90%, even more preferably at least 95%, most preferably 99% of the amino acids of the antigen of any of the SEQ ID NOs: 21 to 40.

8. The protective peptide of claim 1, wherein the functional active variant of the antigen of any of the SEQ ID NOs: 21 to 40 has at least 50% sequence identity to the antigen of any of the SEQ ID NOs: 21 to 40, especially at least 60%, preferably at least 70%, more preferably at least 80%, still more preferably at least 90%, even more preferably at least 95%, most preferably 99% sequence identity to the antigen of any of the SEQ ID NOs: 21 to 40.

9. The protective peptide of claim 8, wherein the variant is derived from the antigen of any of the SEQ ID NOs: 21 to 40 by at least one conservative amino acid substitution.

10. A nucleic acid coding for the protective peptide according to claim 1 or a nucleic acid complementary thereto, particularly a DNA sequence of any of the sequences of SEQ ID NOs: 1 to 20 or the corresponding RNA sequence.

11. The nucleic acid of claim 10, wherein the nucleic acid is located in a vector.

12. A pharmaceutical composition comprising
(i) at least one protective peptide according to claim 1 and
(ii) optionally a pharmaceutically acceptable carrier or excipient.

13. A pharmaceutical composition containing
(i) a nucleic acid according to claim 10 and/or a nucleic acid complementary thereto and
(ii) optionally a pharmaceutically acceptable carrier or excipient.

14. The pharmaceutical composition of claim 13, wherein the nucleic acid is comprised in a vector and/or a cell.

15. An antibody or functional active fragment thereof which binds specifically to the protective peptide according to claim 1.

16. The antibody or functional active fragment thereof of claim 15, wherein the antibody is a monoclonal, polyclonal, chimeric or humanized antibody, or wherein the functional active fragment comprises a Fab fragment.

17. A hybridoma cell line which produces the antibody according to claim 15.

18. A method for producing an antibody comprising:
(a) administering an effective amount of the peptide according to claim 1 to an animal; and
(b) isolating the antibody produced by the animal in response to the administration of step (a) from the animal.

19. A method for producing an antibody comprising:
(a) contacting a B cell with an effective amount of the peptide according to claim 1;
(b) fusing the B cell of step (a) with a myeloma cell to obtain a hybridoma cell; and
(c) isolating the antibody produced by the cultivated hybridoma cell.

20. The method of claim 18, wherein the isolated antibody is additionally purified.

21. A pharmaceutical composition comprising the antibody according to claim 15.

22. A pharmaceutical composition as defined in claim 12 for the immunization of a subject against an infection or the treatment of a subject having an infection.

23. A method for immunization against or treatment of an infection, comprising administering an effective amount of a peptide according to claim 1 to a subject in need thereof.

24. A method for immunization against or treatment of an infection comprising administering an effective amount of an antibody or functional fragment thereof according to claim 15 to a subject in need thereof.

25. A method of diagnosing an E. faecalis infection comprising the steps of:
(a) contacting a sample obtained from a subject with the peptide according to claim 1; and
(b) detecting the presence of an antibody against E. faecalis in the sample.

26. A method of diagnosing an E. faecalis infection comprising the steps of:
(a) contacting a sample obtained from a subject with the antibody according to claim 15; and
(b) detecting the presence of an antigen of E. faecalis in the sample.

27. A method for isolation and/or purification and/or identification of an interaction partner comprising contacting a peptide according to claim 1 with a sample and isolating and/or purifying and/or identifying an interaction partner of the peptide.
